SEQUENCE LISTING (12 sequences)

SEQ ID NO: 1 - protein, 335 aa, synthetic construct
MGHPPLLPLLLLLHTCVPASWGLRCMQCKTNGDCRVEECALGQDLCRTTIVRLWEEGEELELVEKSCTHSEKTNRTLSYRTGLKITSLTEVVCGLDLCNQGNSGRAVTYSRSRYLECISCGSSDMSCERGRHQSLQCRSPEEQCLDVVTHWIQEGEEGRPKDDRHLRGCGYLPGCPGSNGFHNNDTFHFLKCCNTTKCNEGPILELENLPQNGRQCYSCKGNSTHGCSSEETFLIDCRGPMNQCLVATGTHEPKNQSYMVRGCATASMCQHAHLGDAFSMNHIDVSCCTKSGCNHPDLDVQYRSGAAPQPGPAHLSLTITLLMTARLWGGTLLWT

SEQ ID NO: 2 - protein, 99 aa, synthetic construct
MGHPPLLPLLLLLHTCVPASWGLRCMQCKTNGDCRVEECALGQDLCRTTIVRLWEEGEELELVEKSCTHSEKTNRTLSYRTGLKITSLTEVVCGLDLCN

SEQ ID NO: 3 - protein, 17 aa, synthetic construct
QGNSGRAVTYSRSRYLE

SEQ ID NO: 4 - protein, 13 aa, synthetic construct
LWEEGEELELVEK

SEQ ID NO: 5 - protein, 84 aa, synthetic construct
CISCGSSDMSCERGRHQSLQCRSPEEQCLDVVTHWIQEGEEGRPKDDRHLRGCGYLPGCPGSNGFHNNDTFHFLKCCNTTKCNE

SEQ ID NO: 6 - protein, 23 aa, synthetic construct
SPEEQCLDVVTHWIQEGEEGRPK

SEQ ID NO: 7 - protein, 103 aa, synthetic construct
GPILELENLPQNGRQCYSCKGNSTHGCSSEETFLIDCRGPMNQCLVATGTHEPKNQSYMVRGCATASMCQHAHLGDAFSMNHIDVSCCTKSGCNHPDLDVQYR

SEQ ID NO: 8 - protein, 32 aa, synthetic construct
SGAAPQPGPAHLSLTITLLMTARLWGGTLLWT

SEQ ID NO: 9 - protein, 14 aa, synthetic construct
GPILELENLPQNGR

SEQ ID NO: 10 - protein, 17 aa, synthetic construct
RGPMNQCLVATGTHEPK

SEQ ID NO: 11 - protein, 13 aa, synthetic construct
SGCNHPDLDVQYR

SEQ ID NO: 12 - protein, 207 aa, synthetic construct
QGNSGRAVTYSRSRYLECISCGSSDMSCERGRHQSLQCRSPEEQCLDVVTHWIQEGEEGRPKDDRHLRGCGYLPGCPGSNGFHNNDTFHFLKCCNTTKCNECNEGPILELENLPQNGRQCYSCKGNSTHGCSSEETFLIDCRGPMNQCLVATGTHEPKNQSYMVRGCATASMCQHAHLGDAFSMNHIDVSCCTKSGCNHPDLDVQYR

(19) United States
(12) Patent Application Publication
(10) Pub. No.: US 2025/0296999 A1
(43) Pub. Date: Sep. 25, 2025

(54) Novel Treatment of Diabetes and Kidney Disease by Inhibition of D2D3 a Proteolytic UPAR

(71) Applicants: RUSH UNIVERSITY MEDICAL CENTER, Chicago, IL (US); MASSACHUSETTS GENERAL HOSPITAL, Boston, MA (US)

(72) Inventors: Jochen Reiser, Chicago, IL (US); David Changli WEI, Hinsdale, IL (US); Sanja SEVER, Brookline, MA (US); Kamalika MUKHERJEE, Malden, MA (US)

(21) Appl. No.: 18/843,497

(22) PCT Filed: Mar. 7, 2023
(86) PCT No.: PCT/US2023/063815

§ 371 Date: Sep. 3, 2024

Related U.S. Application Data
(60) Provisional application No. 63/317,198, filed on Mar. 7, 2022.

(51) Int. Cl. C07K 16/28; A61P 3/10; G01N 33/68

(52) U.S. Cl. CPC C07K 16/28; A61P 3/10; G01N 33/6893; C07K 2317/24; G01N 2333/9723

(57) ABSTRACT

The current invention discloses methods for treating diseases characterized by elevated levels of the urokinase plasminogen activator receptor (uPAR) protein D2D3 wherein the disease is one or more of chronic kidney disease, insulin-dependent diabetes, or diabetic neuropathy. In addition, the invention provides methods for restoring pancreatic β-cell number and function in the pancreas of a subject diagnosed with insulin-dependent diabetes wherein the insulin-dependent diabetes is characterized by the presence of detectable levels of D2D3 Specifically, the methods comprise administration of a therapeutically effective amount of an agent that antagonizes or removes D2D3 from the circulation of the subject wherein the agent comprises an anti-D2D3 antibody or antigen binding fragment thereof that specifically binds to a D2D3 protein. Alternatively, the methods comprise removing the D2D3 protein from the circulation of the subject by an extracorporeal procedure.

### REFERENCE TO GOVERNMENT GRANTS

This invention was made with government support under several grants awarded by National Institutes of Health including 1RO1DK101350, 1RO1DK125858, 1RO1DK093773 and 1RO1DK087985. The government has certain rights.

### FIELD OF THE INVENTION

The incurrent invention relates to novel treatments for chronic kidney diseases and diabetes.

### BACKGROUND OF THE INVENTION

Chronic kidney diseases (CKDs) affect hundreds of millions of people worldwide. Three common causes of CKD are diabetes mellitus. hypertension, and glomerulonephritis. See Romagnani et al., “Chronic kidney disease,” (2017) Nat Rev Dis Primers 3: p. 17088. Diabetic nephropathy (DN) occurs in approximately 40% of diabetic patients and is a major cause of end-stage renal disease (ESRD) worldwide. See Sulaiman, “Diabetic nephropathy: recent advances in pathophysiology and challenges in dietary management,” (2019) Diabetol Metab Syndr 11: p. 7. The pathogenesis of DN encompasses diverse molecular mechanisms that include genetic, metabolic, and hemodynamic factors such as glomerular hypertrophy and hypertension. Id. Histological and genetic data strongly implicates podocyte dysfunction in the development of DN. See Dai et al., “Research Progress on Mechanism of Podocyte Depletion in Diabetic Nephropathy, (2017) J Diabetes Res 2615286.

Elevated levels of soluble urokinase-type plasminogen activator receptor (suPAR) have been associated with DN in patients with Type 1 diabetes (TID) and Type 2 diabetes (T2D). See Theilade et al . . . “Increased plasma concentrations of midregional proatrial natriuretic peptide is associated with risk of cardiorenal dysfunction in type 1 diabetes,” (2015) Am J Hypertens 28: pp. 772-779; Guthoff et al . . . “Soluble urokinase receptor (suPAR) predicts microalbuminuria in patients at risk for type 2 diabetes mellitus,” (2017) Sci Rep 7: p. 40627; Haugaard et al., “The immune marker soluble urokinase plasminogen activator receptor is associated with new-onset diabetes in non-smoking women and men,” (2012) Diabet Med 29: pp. 479-487; Eugen-Olsen et al., “Circulating soluble urokinase plasminogen activator receptor predicts cancer, cardiovascular disease, diabetes and mortality in the general population,” (2010) J Intern Med 268: pp. 296-308 (2010).

suPAR is generated by proteolytic shedding of the membrane-bound uPAR from the surface of various cells of the innate immune system such as macrophages, immature myeloid cells, and neutrophils. (FIG. 12) See also Andolfo et al., “Metalloproteases cleave the urokinase-type plasminogen activator receptor in the D1-D2 linker region and expose epitopes not present in the intact soluble receptor,” (2002) Thromb Haemost 88: pp. 298-306; van Veen et al., “Negative regulation of urokinase receptor activity by a GPI-specific phospholipase C in breast cancer cells,” (2017) Elife 6. The inventors and others have shown that uPAR/suPAR causes CKD by activating αᵥβ₃ integrin on glomerular podocytes, leading to foot process (FP) effacement and proteinuria. See Kugler et al., “Urokinase receptor and integrin interactions,” (2003) Curr Pharm Des 9: pp. 1565-1574; Castillo et al., “Disruption of thyroid hormone activation in type 2 deiodinase knockout mice causes obesity with glucose intolerance and liver steatosis only at thermoneutrality,” (2011) Diabetes 60: pp. 1082-1089; Dong et al., “Multiple myeloma with a previous diagnosis of focal segmental glomerulosclerosis: A case report and review of the literature.” (2015) Oncol Lett 10: pp. 2821-2827; Alfano et al., “Full-length soluble urokinase plasminogen activator receptor down-modulates nephrin expression in podocytes,” Sci Rep 5: p. 13647; Hahm et al., “Bone marrow-derived immature myeloid cells are a main source of circulating suPAR contributing to proteinuric kidney disease, (2017) Nat Med 23: pp. 100-106.

It is known that uPAR/suPAR consists of three homologous domains: D1, D2, and D3. uPAR proteolysis generates two additional circulating forms: an N-terminal DI fragment and a C-terminal D2D3 protein, both of which are implicated in cancer biology. (FIG. 12) See also Thuno et al., “suPAR: the molecular crystal ball,” (2009) Dis Markers 27: pp. 157-172; Sidenius et al., “Shedding and cleavage of the urokinase receptor (uPAR): identification and characterization of uPAR fragments in vitro and in vivo,” (2000) FEBS Lett 475: pp. 52-56. It is also known that the D2D3 protein induces chemotaxis of cancer cells in part by activating αᵥβ₃ integrin. See Thuno et al. 2009; Aznavoorian et al., “Integrin αᵥβ₃ mediates chemotactic and haptotactic motility in human melanoma cells through different signaling pathways,” (1996) J Biol Chem 271: pp. 3247-3254. Furthermore, increased expression of β₃ integrin has been associated with diabetic nephropathy (DN). See Wilson et al., “The single-cell transcriptomic landscape of early human diabetic nephropathy.” (2019) Proc Natl Acad Sci USA 116: pp. 19619-19625.

In addition, it has been disclosed that suPAR antagonists could be beneficial for the treatment of acute kidney injury. See WO2020069498, the disclosure of which is incorporated by reference in its entirety.

However, the little is known concerning the role of D2D3 in chronic kidney disease, advanced insulin-dependent diabetes. and the potential for D2D3 as a therapeutic target in those diseases. The current application addresses these needs.

### SUMMARY OF THE INVENTION

The current invention is based upon the discovery of the presence of the C-terminal uPAR protein, D2D3, in patients with diabetic nephropathy. D2D3-positive human sera inhibited glucose-stimulated insulin release in human islets and were associated with patients requiring insulin therapy. D2D3 transgenic mice presented kidney disease marked by proteinuria and foot processes effacement. In addition, D2D3 transgenic mice developed diabetes marked by decreased levels of insulin and C-peptide, impaired glucose-stimulated insulin secretion, decreased pancreatic β-cell mass, and high fasting glucose. Recombinant D2D3 protein dysregulated glucose-induced cytoskeletal dynamics, impaired maturation and trafficking of insulin granules. and inhibited bioenergetics of β-cells in culture. An anti-uPAR antibody restored β-cell function and numbers in D2D3 transgenic mice. The current invention identifies the causal role of D2D3 protein in the development of insulin-dependent diabetes mellitus via impaired insulin release and decrease in β-cell numbers in pancreas in the post-natal period. In addition. D2D3 protein also directly injures podocytes in the kidney glomerulus, causing kidney diseases. Detecting D2D3 protein is of clinical value for risk stratification of kidney patients as well as diabetics. The current invention demonstrates that blocking D2D3 protein in circulation ameliorates insulin-dependent diabetes and kidney diseases, thus establishing a unique dual therapeutic approach for kidney diseases and insulin-dependent diabetes. Specifically. the current invention provides methods for treating D2D3-dependent diabetes mellitus and D2D3-dependent kidney disease based upon a novel molecular diagnostic and mechanism.

Thus, the current invention discloses methods for treating chronic kidney disease comprising measuring or having measured the presence of D2D3 protein in a biological sample from the subject and if the presence of D2D3 protein is detected. administering a therapeutically effective amount of an agent that antagonizes D2D3 and/or removes D2D3 from the circulation of the subject. In some embodiments, the agent can be an antibody or antibody fragment. In embodiments, the agent comprises an anti-D2D3 antibody or antigen-binding fragment thereof that specifically binds to a D2D3 protein. In some embodiments the anti-D2D3 antibody or antigen-binding fragment thereof that specifically binds to a D2D3 protein is humanized. In other embodiments, the anti-D2D3 antibody or antigen-binding fragment thereof that specifically binds to a D2D3 protein is a monoclonal antibody. In other embodiments, it is contemplated that D2D3 may be removed from the circulation by an extracorporeal process such as plasmapheresis, dialysis, or immunoadsorption.

In any embodiment, the chronic kidney disease can be caused by diabetes mellitus, hypertension, or glomerulonephritis and is indicative of the presence of D2D3.

The current invention also provides methods for treating any form of insulin-dependent diabetes or its consequences such as diabetes neuropathy comprising measuring or having measured the presence of D2D3 protein in a biological sample from the subject and if the presence of D2D3 protein is detected, administering a therapeutically effective amount of an agent that antagonizes D2D3 and/or removes D2D3 from the circulation of the subject. In some embodiments, the agent can be an antibody or antibody fragment. In embodiments, the agent comprises an anti-D2D3 antibody or antigen-binding fragment thereof that specifically binds to a D2D3 protein. In some embodiments the anti-D2D3 antibody or antigen-binding fragment thereof that specifically binds to a D2D3 protein is humanized. In other embodiments, the anti-D2D3 antibody or antigen-binding fragment thereof that specifically binds to a D2D3 protein is a monoclonal antibody. In other embodiments, it is contemplated that D2D3 may be removed from the circulation by a process such as plasmapheresis or immunoadsorption.

In yet other embodiments, the current invention provides methods to restore both the number of β-cells in the pancreas of patients with insulin-dependent diabetes in which the presence of D2D3 has been detected. The methods comprise measuring or having measured the presence of D2D3 protein in a biological sample from the subject and if the presence of D2D3 protein is detected, administering a therapeutically effective amount of an agent that antagonizes D2D3 and/or removes D2D3 from the circulation of the subject. In some embodiments, the agent can be an antibody or antibody fragment. In embodiments, the agent comprises an anti-D2D3 antibody or antigen-binding fragment thereof that specifically binds to a D2D3 protein. In some embodiments the anti-D2D3 antibody or antigen-binding fragment thereof that specifically binds to a D2D3 protein is humanized. In other embodiments, the anti-D2D3 antibody or antigen-binding fragment thereof that specifically binds to a D2D3 protein is a monoclonal antibody. In other embodiments, it is contemplated that D2D3 may be removed from the circulation by an extracorporeal process such as plasmapheresis, dialysis, or immunoadsorption.

In any of the embodiments, the level of D2D3 is measured by any method known to those of skill in the art, such as mass spectrometry to detect specifically D2D3 in a biological sample, immunoprecipitation coupled to Western Blot analysis or a D2D3-specific ELISA.

In still other embodiments, the methods further comprise the administration of an anti-soluble urokinase plasminogen activator receptor (suPAR) antibody or antibody fragment.

### BRIEF DESCRIPTION OF THE DRAWINGS

FIG. 1A-H depicts that the D2D3 protein discriminates between DN patients on insulin therapy and those that are not. (FIG. 1A) Schematic of human sample analyses. (FIG. 1B and FIG. 1C) suPAR and D2D3 protein detection by IP-WB in DN patient sera (FIG. 1B) or urine (FIG. 1C). Controls: recombinant human suPAR or chymotrypsin digested-suPAR representing D2D3 protein (FIG. 1D) Study patients' characteristics. (FIG. 1E) Comparison (area under the curve, (AUC) values) between patients who were or were not on insulin therapy. (FIG. 1F) Pie chart showing the distribution (%) of D2D3-positive and D2D3-negative sera in DN patients who were or were not on insulin therapy. (FIG. 1G) Scatter dot plots representing levels of GSIS from human islets in the presence of human sera (n=3 experiments). Healthy donor islets were incubated in 10% healthy sera (HS), D2D3-positive sera (D2D3 PS, pooled from 8 patients), D2D3-negative sera (D2D3 NS, pooled from 6 patients), or D2D3 PS that were immunodepleted using an anti-uPAR antibody (R4) (D2D3 DS). When indicated, 10 ng/ml of recombinant hD2D3 or suPAR were added. (FIG. 1 FIG. 1H) Scatter dot plots showing activation of b3 integrin on human podocytes (cells>35) treated with sera as described in (G), excluding hD2D3 concentration of 2.5 ng/ml. Immunofluorescence analysis was performed using an anti-paxillin antibody (focal adhesions), and an AP5 antibody (activated β3 integrin). FIG. 1G and FIG. 1H, error bar, mean±SEM (*P<0.05, **P<0.01, ***P<0.001, ****P<0.0001, unpaired/-test). ns, not significant.

FIG. 2A-J shows that D2D3-Tg mice present glomerular injury and insulin insufficiency. All animals were fed a high-fat diet (HFD) and measurements were performed on 6 months old animals unless stated differently. (FIG. 2A) Schematics of D2D3-Tg construction. (FIG. 2B) qPCR analysis of D2D3 and D1 in fat tissues normalized to Gapdh (n=3 biological replicates). (FIG. 2C) D2D3 detection in fat tissue of D2D3-Tg mice using anti-Myc antibody (D2D3 is Myc tagged). Controls: tissues stained with primary or secondary antibody only. (FIG. 2D) Mouse serum suPAR and D2D3 protein levels were determined using uPAR-specific ELISA (n=8). (FIG. 2E) Detection of Myc-tagged D2D3 in mouse sera. Sera were IPed with anti-uPAR antibody and detected by WB using anti-Myc antibody. Control: recombinant Myc-tagged mD2D3 (Lane 3,4,7,8; +/−N-glycanase treated). (FIG. 2F) Serum IL-6 (WT, n=11; D2D3-Tg, n=7) and CPR (n=7, each group) levels. (FIG. 2G) Fasting blood glucose (n=9), each group), C-peptide (n=6 for WT; n=10 for D2D3-Tg), and insulin (n=7 for WT; n=8 for D2D3-Tg) levels. (FIG. 2H) In vivo GSIS. Overnight fasted animals were intraperitoneally injected with glucose (2 g/kg body weight) and their blood insulin levels were measured by ELISA (n=6-8 for each group). (FIG. 2I) Scatter dot plots presenting albumin creatinine ratio (ACR) or serum creatinine (n=7-10 for each group) levels. (FIG. 2J) PAS-stained kidney (left) and TEM of foot processes (right). (FIG. 2C. FIG. 2J). For all results, error bar, mean±SEM, (*P<0.05; ** P<0.01; ***P<0.001. unpaired/-test). ns, not statistically significant.

FIG. 3A-N shows D2D3-Tg mice present insulin-dependent diabetes due to impaired pancreas function and β-cell mass. All animals were fed a regular diet. (FIG. 3A) Scatter dot plots representing fasting C-peptide and blood insulin levels (n=5-10 mice in each group) (FIG. 3B) In vivo GSIS. Overnight fasted animals were intraperitoneally injected with glucose (2 g/kg body weight) and their blood insulin levels were measured by ELISA (WT, n=5; D2D3-Tg, n=8). (FIG. 3C) In vitro GSIS. Islets from 2-month-old animals (n=3) were subjected to in vitro GSIS. (FIG. 3D) Glucose tolerance test (GTT). Adult (5-6 months) and aged (12 months) mice were fasted overnight before intraperitoneally injected with glucose (2 g/kg body weight). Blood glucose was measured at the indicated time points (n=5-6 animals per each condition). (FIG. 3E) Scatter dot plots depicting fasting blood glucose levels (n=8-10 mice per condition). (FIG. 3F) Representative immunohistochemistry of islets from 2-month-old mice stained with anti-CD4, anti-CD8, and anti-B220 antibodies. Spleen was used as a positive control for T and B cells staining (n=4 mice per each condition). (FIG. 3G) Representative immunohistochemistry of pancreas from 2-month-old mice stained with anti-insulin antibody (n=5 mice per each condition). (FIG. 3H) Representative immunohistochemistry of islets using anti-glucagon (α-cell) and anti-insulin (β-cell) antibodies (n=5 mice per each condition). (FIG. 3I) Scatter dot plots representing β-cell mass and β-cell area/pancreatic area ratio. Data were generated using images shown in (FIG. 3H). When indicated, D2D3-Tg mice were treated with anti-uPAR-Ab or IgG isotype control (IgG) for four weeks beginning at 2 months of age. Those data were collected at 3-month-old animals. (n=5 for WT and D2D3-Tg; n=6 for D2D3-Tg treated with either IgG or uPAR-Ab). Between 6-8 sections from each tissue were analyzed. (FIG. 3J) Scatter dot plots representing the composition of the islets in animals treated as described in (FIG. 3I). Islet composition was determined by counting the total number of β-cells (green) and α-cells (red) and expressing them as percentages of total cells counted within the single islet. (FIG. 3K) Scatter dot plots depicting levels of apoptosis determined by TUNEL staining, or cell proliferation determined by Ki67-positive staining of mice islets (n=5 mice for each condition). Each dot represents an average of 1000-1350 insulin-positive cells counted per animal. (FIG. 3L) In vivo GSIS using two months old D2D3-Tg treated with either IgG or anti-uPAR-Ab twice within one week. All animals were male (n=7-10 animals for each condition). (FIG. 3M and FIG. 3N) D2D3-Tg mice were treated with either anti-uPAR-Ab or IgG beginning at 2 months of age for four weeks. Experiments were performed at 3 months of age. All animals were male. Scatter dot plots representing blood insulin levels are shown in (FIG. 3M) (n=8 mice for each condition). The glucose tolerance test is shown in (FIG. 3N) (n=6 mice for each condition). For all results, error bar, mean±SEM, (*P<0.05; ** P<0.01; *** P<0.001. unpaired/-test). ns, not statistically significant. In addition, when appropriate P numbers are shown in red or blue.

FIG. 4A-L shows that D2D3 protein impairs multiple aspects of β-cell physiology. (FIG. 4A) Graphs depicting GSIS by MIN-6 cells were incubated in the presence or absence of BSA, mD2D3 (100 ng/ml). and anti-uPAR Ab (n=3). (FIG. 4B) Graphs depicting GSIS by mouse islets were incubated in the presence or absence of BSA, mD2D3 (100 ng/ml), and anti-uPAR Ab (n=3). (FIG. 4C) Graphs depicting GSIS by human islets were incubated in the presence or absence of BSA, hD2D3 (100 ng/ml) (n=3). (FIG. 4D) PR-EM micrographs showing the cytoskeleton of MIN6 cells. MIN6 cells were grown as described in (FIG. 4A) with exception of increasing glucose levels to 20 nM. When indicated, an mD2D3 protein (100 ng/ml) was added for 24 hours. Actin bundles (pink), hyper bundles (purple), and microtubules (MT, green). (FIG. 4E) TEM micrographs of MIN-6 cells were treated as described in (D). Large dense-core vesicles (LDCVs) localized near (yellow) the plasma membrane (pink) and those localized more than 500 nm away (green) from the plasma membrane. (FIG. 4F and FIG. 4G) Graphs represent average LDCV diameter (FIG. 4F), number of LDCV per cell, and distribution based on location (FIG. 4G) in cells shown in (FIG. 4E). In (F), more than 75 vesicles were counted in ≥10 cells per treatment. Of note, the overall number of LDCV per cell regardless of the treatment was similar and ranged between 17-57. Error bar, mean±SD. (FIG. 4H) Schematic for the experimental setup for glycolysis and the oxygen consumption rate (OCR) measurement in MIN-6 cells. (FIG. 4I-J) Extracellular acidification rates (ECAR) were measured in untreated (CTL) and D2D3-treated cells using the Seahorse XFe24 analyzer. When indicated, cells were treated with hD2D3. (FIG. 4K-L) OCR curves and measurements of the bioenergetic parameters regarding mitochondrial respiration of the treated cells as explained in (H). Each OCR value was normalized to cell number and presented as pmol/min/100,000 cells (three experiments). For FIG. 4J and FIG. 4L Error bars mean±S.E.M. (*P<0.05, **P<0.01, ***P<0.001, one-way ANOVA). ns, not statistically significant.

FIG. 5A,B shows uPAR-specific peptides in human samples. (FIG. 5A) Amino acid sequence of human uPAR protein isoform 1 (SEQ ID NO: 1). Distinct domains are labeled, Domain 1 (D1), Domain 2 (D2), and Domain 3 (D3). Linker between D1 and D2, and the GPI anchor underlined and labeled. Bold letters demarcate suPAR-specific peptides detected by mass spectrometry, the details of which are shown herein. Specifically shown are Fragments-1 to Fragment-5. * Additional fragment generated within the Fragment-4. Fragment-3 spans over D2 and D3 domain and thus is marked 3/3. (FIG. 5B) Coomassie gel showing proteins immunoprecipitated from sera using anti-uPAR antibody (R4). Serum samples (10 ml) from 14 DN patients that also had a D2D3-like fragment in their sera were used in the experiment. The protein band indicated in the figure was cut out of the gel and analyzed using mass spectrometry.

FIG. 6A-D shows that recombinant hD2D3 protein activates β₃ integrin on human podocytes. (FIG. 6A) Representative images of human podocytes stained with phalloidin (F-actin) and AP5 (an antibody that recognizes active form of β₃ integrin). Notice that serum free media (SFM) and healthy serum (HS) do not activate β3 integrin, but that the addition of hD2D3 (2.5 ng/ml) to the HS resulted in AP5 signal. Scale bar, 5 μm. (FIG. 6B) Graph showing concentration-dependence of hD2D3's ability to activate β₃ integrin on human podocytes grown in the presence of healthy human serum. 20-101 cells were analyzed per treatment. Error bar=mean±SEM. Notice that at lower protein concentrations (1−˜3 ng/ml) activation appears cooperative. (FIG. 6C) Scatter dot plots depicting ratio between activated β₃ integrin (AP5) and overall focal adhesions determined by the paxillin staining. Notice that addition of hD2D3 to SFM activated β3 integrin on human podocytes. This demonstrates that hD2D3 activates β₃ integrin on human podocytes even in the absence of any other serum proteins. For experiments with SFM, 62-79 cells were analyzed. For experiments with HS, 43-46 cells were analyzed. Data are shown as mean±SEM. An unpaired two-tailed/-test was performed to determine statistical significance (****P<0.0001). (FIG. 6D) Scatter dot plots showing levels of activated β3 integrins on human podocytes (38-53 cells) treated with 10% healthy sera (HS), D2D3-positive sera (D2D3 PS), D2D3-negative sera (D2D3 NS), or D2D3 PS that were immunodepleted using an anti-uPAR antibody (R4) (D2D3 DS). Where indicated, 2.5 ng/ml hD2D3 was added. Data are presented relative to the control (HS or SFM). All results are presented as mean 1 SEM. Statistical significance was assessed using one-way ANOVA with Tukey's multiple comparisons.

FIG. 7A-C shows that ELISA detects mouse suPAR and mD2D3 protein. (FIG. 7A) Coomassie gel showing recombinant mouse suPAR and mD2D3 protein that were expressed and purified from HEK-293 cells. Notice that protein purification resulted in highly pure proteins. As both proteins are glycosylated, they travel as wide bands. (FIG. 7B) Table summarizing MWs of mouse proteins. MW of mD2D3 detected in the plasma of D2D3-Tg mice was similar to the MW of purified recombinant protein. (FIG. 7C) Mouse-specific ELISA (R&D) detected full length suPAR and mD2D3 equally well, but with low sensitivity. BCA was used to determine protein concentrations used in ELISAs.

FIG. 8A-I shows that D2D3-Tg mice on a regular diet do not develop glomerular injury. (FIG. 8A) Scatter dot plot showing fasting blood insulin levels in WT and mice expressing mouse suPAR isoform 1 (suPAR-Tg) (n=15 for each group). (FIG. 8B) Scatter dot plots comparing body weights of animals kept on regular diet and on high fat diet (HFD). Notice that expression of mD2D3 had no effect on the body weight of the animals regardless of their diet. (FIG. 8C) qPCR analysis of D2D3 and D1 in fat tissues. D2D3 and D1 mRNA levels were normalized to Gapdh mRNA level (n=3 biologic replicates). Data show that mRNA for D2D3 was detected in the fat tissue even when animals were fed regular diet. (FIG. 8D) mD2D3 protein is expressed in fat tissue of animals that were fed regular diet. As D2D3-Tg carries the c-Myc tag, immunohistochemistry was performed with a rabbit anti-Myc antibody. D2D3 was observed in adipocytes of D2D3-Tg mice, but not in control mice. Tissues stained with only primary or secondary antibody were used as negative controls. Scale bar. 50 μm. (FIG. 8E) Levels of suPAR and suPAR/D2D3 increased as animals aged. Scatter dot plots depicting protein levels determined using mouse-specific ELISA (n=5-11 for each group). (FIG. 8F) Body weight of WT and D2D3-Tg mice (n=5-8 for each group). (FIG. 8G) Kidney function was not affected in D2D3-Tg mice when they were fed regular diet. Kidney function was assessed by measuring ACR, serum creatinine and serum BUN levels (n=5-12 for each group). (FIG. 8H) Immunohistochemistry of islets using anti-CD4 and anti-CD8 antibodies in 12-months old animals. (FIG. 8I) Scatter dot plots comparing pancreatic weight between WT (n=5) and D2D3-Tg (n=5), as well as D2D3-Tg animals treated with IgG (n=6) or anti uPAR-Ab (n=6). All results were expressed as mean±SEM. Statistical analysis was determined using 2-tailed unpaired Student's/test. *P<0.05; **P<0.01;***P<0.001. ns, not significant.

FIG. 9A-D shows that neonatal D2D3-Tg mice have normal β-cell mass. (FIG. 9A) Immunohistochemistry of pancreas isolated from the neonatal mice (P0) stained with anti-insulin and anti-glucagon antibodies. (FIG. 9B) Staining shown in A was used to determine the β-cell area/pancreatic area ratio (n=5, for each group) as well as β-cell mass (n=5 for each group). (FIG. 9C) Scatter dot plots showing Islet composition (n=5, for each group) and pancreatic weight (n=5, for each group) in neonatal mice. (FIG. 9D) GSIS of MIN6 cells cultured in the presence or absence of BSA (100 ng/ml), IgG (100 ng/ml), mD2D3 protein or mouse suPAR (n=3). All results are expressed as mean 1 SEM. Statistical analysis was determined using 2-tailed unpaired Student's/test. *P<0.05; **P<0.01; ***P<0.001. ns, not significant.

FIG. 10A,B shows that mD2D3 fragment impairs glucose-stimulated reorganization of the actin cytoskeleton in MIN6 cells. (FIG. 10A) MIN6 cells were grown in low glucose (5 mM) before being stimulated by the addition of high glucose (20 mM) in the presence or absence of mD2D3 protein (100 ng/ml). The status of F-actin was examined by phalloidin staining. (FIG. 10B) Representative PR-EM micrographs of MIN6 cells focusing on the organization and the status of the actin cytoskeleton. Cells were grown as described in (FIG. 10A) Notice that mD2D3 inhibited high glucose-induced disassembly of actin filaments.

FIG. 11A-C shows that mD2D3 impairs insulin granule maturation and trafficking. Representative TEM micrographs of MIN6 cells. When indicated cells were grown in low glucose (5 mM) before stimulating by the addition of a high glucose (5 mM) in the presence or absence of mD2D3 protein (100 ng/ml) for 24 h. Images shown were captured at increasing magnifications. The plasma membrane is colored pink and large dense core vesicles (LDCVs) that transport insulin are colored yellow. Notice that LDCVs are smaller in size and less associated with the plasma membrane in MIN6 cells grown in the presence of the mD2D3 protein.

FIG. 12 is a schematic depicting proteolytic shedding of the membrane-bound uPAR into suPAR, the D1 and D2D3 proteins.

### DETAILED DESCRIPTION OF THE INVENTION

The current invention discloses methods for treating chronic kidney disease comprising measuring or having measured the presence of D2D3 protein in a biological sample from the subject and if the presence of D2D3 protein is detected, administering a therapeutically effective amount of an agent that antagonizes D2D3 and/or removes D2D3 from the circulation of the subject. In some embodiments, the agent can be an antibody or antibody fragment. In embodiments, the agent comprises an anti-D2D3 antibody or antigen-binding fragment thereof that specifically binds to a D2D3 protein. In some embodiments the anti-D2D3 antibody or antigen-binding fragment thereof that specifically binds to a D2D3 protein is humanized. In other embodiments, the anti-D2D3 antibody or antigen-binding fragment thereof that specifically binds to a D2D3 protein is a monoclonal antibody. In other embodiments, it is contemplated that D2D3 may be removed from the circulation by a process such as plasmapheresis or immunoadsorption.

In any embodiment, the chronic kidney disease can be caused by diabetes mellitus, hypertension, or glomerulonephritis and is indicative of the presence of D2D3.

The current invention also provides methods for treating any form of insulin-dependent diabetes or its consequences such as diabetes neuropathy comprising measuring or having measured the presence of D2D3 protein in a biological sample from the subject and if the presence of D2D3 proteins is detected, administering a therapeutically effective amount of an agent that antagonizes D2D3 and/or removes D2D3 from the circulation of the subject. In some embodiments, the agent can be an antibody or antibody fragment. In embodiments, the agent comprises an anti-D2D3 antibody or antigen-binding fragment thereof that specifically binds to a D2D3 protein. In some embodiments the anti-D2D3 antibody or antigen-binding fragment thereof that specifically binds to a D2D3 protein is humanized. In other embodiments, the anti-D2D3 antibody or antigen-binding fragment thereof that specifically binds to a D2D3 protein is a monoclonal antibody. In other embodiments, it is contemplated that D2D3 may be removed from the circulation by an extracorporeal process such as plasmapheresis, dialysis or immunoadsorption.

In yet other embodiments, the current invention provides methods to restore both the number of β-cells in the pancreas of patients with insulin-dependent diabetes in which the presence of D2D3 has been detected. The methods comprise measuring or having measured the presence of D2D3 protein in a biological sample from the subject and if the presence of D2D3 proteins is detected, administering a therapeutically effective amount of an agent that antagonizes D2D3 and/or removes D2D3 from the circulation of the subject. In some embodiments, the agent can be an antibody or antibody fragment. In embodiments, the agent comprises an anti-D2D3 antibody or antigen-binding fragment thereof that specifically binds to a D2D3 protein. In some embodiments the anti-D2D3 antibody or antigen-binding fragment thereof that specifically binds to a D2D3 protein is humanized. In other embodiments, the anti-D2D3 antibody or antigen-binding fragment thereof that specifically binds to a D2D3 protein is a monoclonal antibody. In other embodiments, it is contemplated that D2D3 may be removed from the circulation by an extracorporeal process such as plasmapheresis, dialysis or immunoadsorption.

In any of the embodiments, the level of D2D3 is measured by any method known to those of skill in the art, such as mass spectrometry to detect specifically D2D3 in a biological sample. immunoprecipitation coupled to Western Blot analysis or a D2D3-specific ELISA.

In still other embodiments, the methods further comprise the administration of an anti-soluble urokinase plasminogen activator receptor (suPAR) antibody or antibody fragment.

### Definitions Used Throughout the Disclosure

The term “antibody” as used herein refers to whole antibodies that interact with (e.g., by binding, steric hindrance, stabilizing/destabilizing, spatial distribution) a D2D3 epitope and inhibit signal transduction. A naturally occurring “antibody” is a glycoprotein comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains. CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. The term “antibody” includes for example, monoclonal antibodies, human antibodies, humanized antibodies, camelized antibodies, chimeric antibodies, single-chain Fvs (scFv), disulfide-linked Fvs (sdFv), Fab fragments, F (ab′) fragments, and anti-idiotypic (anti-Id) antibodies (including, e.g., anti-Id antibodies to antibodies of the invention), and epitope-binding fragments of any of the above. The antibodies can be of any isotype (e.g., IgG, IgE, IgM, IgD, IgA and IgY), class (e.g., IgG1, IgG2, IgG3, IgG4, IgA1 and IgA2) or subclass. Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention the numbering of the constant region domains increases as they become more distal from the antigen binding site or amino-terminus of the antibody. The N-terminus is a variable region and at the C-terminus is a constant region: the CH3 and CL domains actually comprise the carboxy-terminus of the heavy and light chain, respectively.

The phrase “antibody fragment,” as used herein, refers to one or more portions of an antibody that retain the ability to specifically interact with (e.g., by binding, steric hindrance, stabilizing/destabilizing, spatial distribution) a D2D3 epitope and inhibit signal transduction. Examples of binding fragments include, but are not limited to, a Fab fragment, a monovalent fragment consisting of the VL, VH, CL and CHI domains: a F(ab)2 fragment, a bivalent fragment comprising two Fab fragments linked by a disulfide bridge at the hinge region: a Fd fragment consisting of the VH and CHI domains: a Fy fragment consisting of the VL and VH domains of a single arm of an antibody: a dAb fragment (Ward et al. (1989) Nature 341: pp. 544-546), which consists of a VH domain: and an isolated complementarity determining region (CDR).

Furthermore, although the two domains of the Fy fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv). See for example, Bird et al, (1988) Science 242: pp. 423-426; and Huston et al, (1988) Proc. Natl. Acad, Sci, 85: pp. 5879-5883. Such single chain antibodies are also intended to be encompassed within the term “antibody fragment.” These antibody fragments are obtained using conventional techniques known to those of skill in the art, and the fragments are screened for utility in the same manner as are intact antibodies. Antibody fragments can also be incorporated into single domain antibodies, maxibodies, minibodies. intrabodies, diabodies, triabodies, tetrabodies, v-NAR and bis-scFv. See for example Hollinger and Hudson, (2005) Nature Biotechnology 23: pp. 1126-1136. Antibody fragments can be grafted into scaffolds based on polypeptides such as Fibronectin type III (Fn3). See U.S. Pat. No. 6,703,199, which describes fibronectin polypeptide monobodies.

Antibody fragments can be incorporated into single chain molecules comprising a pair of tandem Fv segments (VH-CHI-VH-CH1) which, together with complementary light chain polypeptides: form a pair of antigen binding regions. See for example Zapata et al., (1995) Protein Eng. 8: 1057-1062; and U.S. Pat. No. 5,641,870.

The phrases “monoclonal antibody” or “monoclonal antibody composition” as used herein refers to polypeptides, including antibodies, antibody fragments, bispecific antibodies, etc. that have substantially identical to amino acid sequence or are derived from the same genetic source. This term also includes preparations of antibody molecules of single molecular composition. A monoclonal antibody composition displays a single binding specificity and affinity for a particular epitope.

The phrase “human antibody” or “humanized antibody” as used herein, includes antibodies having variable regions in which both the framework and CDR regions are derived from sequences of human origin. Furthermore, if the antibody contains a constant region, the constant region also is derived from such human sequences, e.g., human germline sequences, or mutated versions of human germline sequences or antibody containing consensus framework sequences derived from human framework sequences analysis, for example, as described in Knappik et al., (2000) J Mol Biol 296: pp. 57-86. The structures and locations of immunoglobulin variable domains, e.g., CDRs, may be defined using well known numbering schemes, e.g., the Kabat numbering scheme, the Chothia numbering scheme, or a combination of Kabat and Chothia. See for example Kabat et al., “Sequences of Proteins of Immunological Interest,” 5th edit., NIH Publication no. 91-3242, U.S. Department of Health and Human Services (1991; Lazikani et al., (1997) J. Mol. Bio. 273: pp. 927-948); Chothia et al., (1987) J. Mol. Biol. 196: pp. 901-917; Chothia et al. (1989) Nature 342: pp. 877-883; Al-Lazikani et al., (1997) J. Mol. Biol. 273; pp. 927-948. The human antibodies of the invention may include amino acid residues not encoded by human sequences (e.g., mutations introduced by random or site-specific mutagenesis in vitro or by somatic mutation in vivo, or a conservative substitution to promote stability or manufacturing).

The phrase “human monoclonal antibody” as used herein refers to antibodies displaying a single binding specificity which have variable regions in which both the framework and CDR regions are derived from human sequences. In one embodiment, the human monoclonal antibodies are produced by a hybridoma which includes a B cell obtained from a transgenic nonhuman animal, e.g., a transgenic mouse, having a genome comprising a human heavy chain transgene and a light chain transgene fused to an immortalized cell.

The phrase “recombinant human antibody” as used herein includes all human antibodies that are prepared, expressed, created or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the human antibody, e.g., from a transfectoma, antibodies isolated from a recombinant, combinatorial human antibody library, and antibodies prepared, expressed, created or isolated by any other means that involve splicing of all or a portion of a human immunoglobulin gene, sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

Specific binding between two entities means a binding with an equilibrium constant (KA) (kₒₙ/k_{off}) of at least 10² M⁻¹, at least 5×10² M⁻¹, at least 10³ M⁻¹, at least 5×10³ M⁻¹, at least 10⁴ M⁻¹ at least 5×10⁴ M⁻¹, at least 10⁵ M⁻¹, at least 5×10⁵ M⁻¹, at least 10⁶ M⁻¹, at least 5×10⁶ M⁻¹, at least 10⁷ M⁻¹, at least 5×10⁷ M⁻¹, at least 10⁸ M⁻¹, at least 5×10⁸ M⁻¹, at least 10⁹ M⁻¹, at least 5×10⁹ M⁻¹, at least 10¹⁰ M⁻¹, at least 5×10¹⁰ M⁻¹, at least 10¹¹ M⁻¹, at least 5×10¹¹ M⁻¹, at least 10¹² M⁻¹, at least 5×10¹² M⁻¹, at least 10¹³ M⁻¹, at least 5×10¹³ M^{31 1}, at least 10¹⁴ M⁻¹, at least 5×10¹⁴ M⁻¹, at least 10¹⁵ M⁻¹, or at least 5×10¹⁵ M¹.

The phrase “specifically (or selectively) binds” to an antibody (e.g., a D2D3 binding antibody) refers to a binding reaction that is determinative of the presence of a cognate antigen (e.g., a human D2D3 protein) in a heterogeneous population of proteins and other biologics. In addition to the equilibrium constant (KA) noted above, a D2D3 binding antibody of the invention typically also has a dissociation rate constant (KD) (k_{off}/kₒₙ) of less than 5×10⁻² M, less than 10⁻² M, less than 5×10⁻³ M, less than 10⁻³ M, less than 5×10⁴ M, less than 10⁻⁴ M, less than 5×10⁵ M, less than 10⁻⁵ M, less than 5×10⁻⁶ M, less than 10⁻⁶ M, less than 5×10⁻⁷ M, less than 10⁻⁷ M, less than 5×10⁻⁸ M, less than 10⁻⁸ M, less than 5×10⁻⁹ M, less than 10⁻⁹ M, less than 5×10⁻¹⁰ M, less than 10⁻¹⁰ M, less than 5×10⁻¹¹ M, less than 10⁻¹¹ M, less than 5×10⁻¹² M, less than 10⁻¹² M, less than 5×10⁻¹³ M, less than 10⁻¹³ M, less than 5×10⁻¹⁴ M, less than 10⁻¹⁴ M, less than 5×10⁻¹⁵ M, or less than 10⁻¹⁵ M or lower, and binds to D2D3 with an affinity that is at least twofold greater than its affinity for binding to a non-specific antigen (e.g., HSA).

In one embodiment, the antibody or fragment thereof has dissociation constant (Ka) of less than 3000 pM, less than 2500 pM, less than 2000 pM, less than 1500 pM, less than 1000 pM, less than 750 pM, less than 500 pM, less than 250 pM, less than 200 pM, less than 150 pM, less than 100 pM, less than 75 pM, less than 10 pM, less than 1 pM as assessed using a method described herein or known to one of skill in the art (e.g., a BIACORE assay, ELISA, FACS, SET) (Biacore International AB, Uppsala, Sweden). The term “K_{assoc}” or “Kₐ”, as used herein, refers to the association rate of a particular antibody-antigen interaction, whereas the term “Kⱼ” or “K_{d},” as used herein, refers to the dissociation rate of a particular antibody-antigen interaction. The term “KD”, as used herein, refers to the dissociation constant, which is obtained from the ratio of Kⱼ to Kₐ (i.e. Kⱼ/Kₐ) and is expressed as a molar concentration (M). KD values for antibodies can be determined using methods well established in the art. A method for determining the KD of an antibody is by using surface plasmon resonance or using a biosensor system such as a BIACORE system.

The term “affinity” as used herein refers to the strength of interaction between antibody and antigen at single antigenic sites. Within each antigenic site, the variable region of the antibody “arm” interacts through weak non-covalent forces with antigen at numerous sites, the more interactions, the stronger the affinity.

The term “avidity” as used herein refers to an informative measure of the overall stability or strength of the antibody-antigen complex. It is controlled by three major factors: antibody epitope affinity; the valence of both the antigen and antibody; and the structural arrangement of the interacting parts. Ultimately these factors define the specificity of the antibody, that is, the likelihood that the particular antibody is binding to a precise antigen epitope.

The term “valency” as used herein refers to the number of potential target binding sites in a polypeptide. Each target binding site specifically binds one target molecule or specific site (i.e, epitope) on a target molecule. When a polypeptide comprises more than one target binding site, each target binding site may specifically bind the same or different molecules (e.g., may bind to different molecules, e.g., different antigens, or different epitopes on the same molecule).

The phrase “antagonist antibody” as used herein refers to an antibody that binds with a D2D3 protein and neutralizes the biological activity of D2D3 signaling. e.g., reduces. decreases and/or inhibits D2D3 induced signaling activity by clearing circulating D2D3 levels in the blood.

The phrase “isolated antibody” refers to an antibody that is substantially free of other antibodies having different antigenic specificities (e.g. an isolated antibody that specifically binds D2D3 or a D2D3 protein is substantially free of antibodies that specifically bind antigens other than D2D3). An isolated antibody that specifically binds D2D3 or a D2D3 protein may, however, have cross-reactivity to other antigens. Moreover, an isolated antibody may be substantially free of other cellular material and/or chemicals.

The phrase “conservatively modified variant” applies to both amino acid and nucleic acid sequences. With respect to particular nucleic acid sequences. “conservatively modified variants” refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA. GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations.” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

For polypeptide sequences, “conservatively modified variants” include individual substitutions, deletions or additions to a polypeptide sequence which result in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. The following eight groups contain amino acids that are conservative substitutions for one another: 1) Alanine (A), Glycine (G); 2) Aspartic acid (D), Glutamic acid (E); 3) Asparagine (N), Glutamine (0); 4) Arginine (R), Lysine (K); 5) Isoleucine (I), Leucine (L), Methionine (M), Valine (V); 6) Phenylalanine (F), Tyrosine (Y), Tryptophan (W); 7) Serine(S), Threonine (T): and 8) Cysteine (C), Methionine (M). See for example Creighton, Proteins (1984). In some embodiments, the term “conservative sequence modifications” are used to refer to amino acid modifications that do not significantly affect or alter the binding characteristics of the antibody containing the amino acid sequence.

The terms “cross-compete” and “cross-competing” are used interchangeably herein to mean the ability of an antibody or other binding agent to interfere with the binding of other antibodies or binding agents to D2D3 in a standard competitive binding assay.

The ability or extent to which an antibody or other binding agent is able to interfere with the binding of another antibody or binding molecule to D2D3, and therefore whether it can be said to cross-compete according to the invention, can be determined using standard competition binding assays. One suitable assay involves the use of the BIACORE technology (e.g. by using the BIACORE 3000 instrument (Biacore, Uppsala, Sweden)), which can measure the extent of interactions using surface plasmon resonance technology. Another assay for measuring cross-competing uses an ELISA-based approach.

The term “optimized” as used herein refers to a nucleotide sequence has been altered to encode an amino acid sequence using codons that are preferred in the production cell or organism, generally a eukaryotic cell, for example, a cell of Pichia, a cell of Trichoderma, a Chinese Hamster Ovary cell (CHO) or a human cell. The optimized nucleotide sequence is engineered to retain completely or as much as possible the amino acid sequence originally encoded by the starting nucleotide sequence, which is also known as the “parental” sequence.

Standard assays to evaluate the binding ability of the antibodies toward D2D3 of various species are known in the art, including for example, ELISAs, western blots and RIAs. The binding kinetics (e.g., binding affinity) of the antibodies also can be assessed by standard assays known in the art, such as by BIACORE analysis, or FACS relative affinity (Scatchard). Assays to evaluate the effects of the antibodies on functional properties of D2D3 known in the art may be used.

The terms “polypeptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. Unless otherwise indicated, a particular polypeptide sequence also implicitly encompasses conservatively modified variants thereof.

“Measuring” or “measurement” means assessing the presence, absence, quantity or amount (which can be an effective amount) of a given substance within a sample, including the derivation of qualitative or quantitative concentration levels of such substances, or otherwise evaluating the values or categorization of a subject's clinical parameters. Alternatively, the term “detecting,” or “detection” may be used and is understood to cover all measuring or measurement as described herein.

The terms “sample” or “biological sample” as used herein, refers to a sample of biological fluid, tissue, or cells, in a healthy and/or pathological state obtained from a subject. Such samples include, but are not limited to, blood, bronchial lavage fluid, sputum, saliva, urine, amniotic fluid, lymph fluid, tissue or fine needle biopsy samples, peritoneal fluid, cerebrospinal fluid, nipple aspirates, and includes supernatant from cell lysates, lysed cells, cellular extracts, and nuclear extracts. In some embodiments, the whole blood sample is further processed into serum or plasma samples. Preferably the biological sample is selected from serum, plasma, saliva and urine. See examples herein and also Fernandez-Botran et al., “The levels of soluble urokinase plasminogen activator receptor (suPAR) in saliva are influenced by acute stress,” (2021) Biological Psychology 165: p. 108147.

“Treating,” “treat,” or “treatment” within the context of the instant invention, means an alleviation of symptoms associated with a disorder or disease, or halt of further progression or worsening of those symptoms, or prevention or prophylaxis of the disease or disorder.

The term Chronic Kidney Disease (CKD) is a chronic and progressive condition that arises when one or both of the following conditions are present: i) when there is evidence of kidney damage lasting for at least 3 months, as defined by structural or functional abnormalities of the kidney with or without a decreased glomerular filtration rate (GFR), as demonstrated either by pathologic abnormalities or by markers of kidney damage, including urine or blood abnormalities or abnormalities noted on imaging: and/or, ii) when the GFR is less than 60 ml/min/1.73 m² for at least 3 months with or without kidney damage. Currently, in the United States, nearly 22% of the adult population has CKD, thereby making it a highly prevalent disease process. CKD is categorized by the level of the GFR and the presence or absence of proteinuria. Stage 1 includes patients with no decrease in GFR but with kidney abnormalities. Stage 2 includes patients with mild CKD with an estimated GFR (eGFR) of 60 to 89 ml/min/1.73 m² and kidney abnormalities. Stage 3 includes patients with an eGFR of 30 to 59 ml/min/1.73 m², and Stage 4 patients have an eGFR of 15 to 29 ml/min/1.73 m². Stage 5 is kidney failure; this includes patients with an eGFR of less than 15 ml/min/1.73 m². After a patient begins dialysis, there is a 1-year mortality rate of approximately 20% and a nearly 75% mortality rate at 5 years. CKD can arise from a myriad of condition, including diabetes (type-1, type-2), hypertension, and glomerulonephritis. In any instance, the presence of D2D3 is indicative of CKD.

As used herein, “Type-1 diabetes” or “insulin-dependent diabetes” refers to is a chronic illness characterized by the body's inability to produce insulin due to the autoimmune destruction of the β-cells in the pancreas. Although onset frequently occurs in childhood. the disease can also develop in adults. Insulin-dependent diabetes is distinguished from “Type-2 diabetes” which refers to an array of dysfunctions characterized by hyperglycemia and resulting from the combination of resistance to insulin action, inadequate insulin secretion, and excessive or inappropriate glucagon secretion. The presence of D2D3 is indicative of insulin-dependent diabetes.

### D2D3 Detection

In some embodiments, the presence of D2D3 in a biological sample is made. The D2D3 determination may be made at any time, for example before a medical procedure or after a medical procedure.

In some embodiments, the presence of D2D3 may be detected from the subject's biological sample. Detection of the presence of D2D3 in the biological sample may be made using any method known to one skilled in the art. Methods for detecting the presence of D2D3 include but are not limited to Enzyme-linked immunosorbent assay (ELISA), Western blot, immunoprecipitation, immunohistochemistry, Radio-immuno Assay (RIA), radioreceptor assay, proteomics methods, mass-spec based detection (SRM or MRM) or quantitative immunostaining methods.

In any embodiment, the presence of D2D3 determines whether or not an agent that antagonizes D2D3 is administered to a subject. In some embodiments, the agent is an anti-D2D3 antibody, or antigen-binding fragment thereof that specifically binds to D2D3. In some embodiments, the anti-D2D3 antibody is a commercially available anti-D2D3 antibody or an antigen-binding fragment thereof that specifically binds to D2D3 or an anti-D2D3 antibody or antigen-binding fragment thereof that specifically binds to D2D3.

### Removal of D2D3

The invention also contemplates that in various embodiments. D2D3 may be removed from the circulation of a patient in which D2D3 has been detected by any extracorporeal means known to those of skill in the art. Such extracorporeal means include but are not limited to plasmapheresis or therapeutic plasma exchange, dialysis, immunoadsorption or any combination of procedures capable of removing D2D3 proteins from the circulation.

### Pharmaceutical Compositions

To prepare pharmaceutical or sterile compositions including D2D3-binding antibodies (intact or binding fragments), the D2D3-binding antibodies (intact or binding fragments) is mixed with a pharmaceutically acceptable carrier or excipient. The compositions can additionally contain one or more other therapeutic agents that are suitable for treating or preventing diabetes.

Formulations of therapeutic and diagnostic agents can be prepared by mixing with physiologically acceptable carriers, excipients, or stabilizers in the form of, e.g., lyophilized powders, slurries, aqueous solutions, lotions, or suspensions (see. e.g., Hardman et al., (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y.).

Selecting an administration regimen for a therapeutic depends on several factors, including the serum or tissue turnover rate of the entity, the level of symptoms, the immunogenicity of the entity, and the accessibility of the target cells in the biological matrix. In certain embodiments, an administration regimen maximizes the amount of therapeutic delivered to the patient consistent with an acceptable level of side effects. Accordingly, the amount of biologic delivered depends in part on the particular entity and the severity of the condition being treated. Guidance in selecting appropriate doses of antibodies, cytokines, and small molecules are available (see, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, N.Y.; Bach (ed.) (1993) Monoclonal Antibodies and Peptide Therapy in Autoimmune Diseases, Marcel Dekker, New York, N.Y.; Baert et al, (2003) New Engl. J. Med. 348:601-608; Milgrom et al, (1999) New Engl. J. Med. 341:1966-1973; Slamon et al, (2001) New Engl. J. Med. 344:783-792; Beniaminovitz et al, (2000) New Engl. J. Med. 342:613-619; Ghosh et al, (2003) New Engl. J. Med. 348:24-32; Lipsky et al. (2000) New Engl. J. Med. 343:1594-1602).

Determination of the appropriate dose is made by the clinician, e.g., using parameters or factors known or suspected in the art to affect treatment or predicted to affect treatment. Generally, the dose begins with an amount somewhat less than the optimum dose and it is increased by small increments thereafter until the desired or optimum effect is achieved relative to any negative side effects.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors known in the medical arts.

Compositions comprising antibodies or fragments thereof of the invention can be provided by continuous infusion, or by doses at intervals of, e.g., one day, one week, or 1-7 times per week. Doses may be provided intravenously, subcutaneously, topically, orally, nasally, rectally, intramuscular, intracerebrally, or by inhalation. A specific dose protocol is one involving the maximal dose or dose frequency that avoids significant undesirable side effects. A total weekly dose may be at least 0.05 p/kg body weight, at least 0.2 pg/kg, at least 0.5 μg/kg, at least 1 pg/kg, at least 10 pg/kg, at least 100 pg/kg, at least 0.2 mg/kg, at least 1.0 mg/kg, at least 2.0 mg/kg, at least 10 mg/kg, at least 25 mg/kg, at least 30 mg/kg, at least 40 mg/kg or at least 50 mg/kg (see, e.g., Yang et al. (2003) New Engl. J. Med. 349:427-434; Herold et al, (2002) New Engl. J. Med. 346:1692-1698; Liu et al, (1999) J. Neurol. Neurosurg. Psych. 67:451-456; Portielji et al, (2003) Cancer Immunol. Immunother. 52:133-144).

The desired dose of antibodies or fragments thereof is about the same as for an antibody or polypeptide, on a moles/kg body weight basis. The desired plasma concentration of the antibodies or fragments thereof is about, on a moles/kg body weight basis. The dose may be at least 15 pg at least 20 pg, at least 25 pg, at least 30 pg, at least 35 pg, at least 40 pg,at least 45 pg, at least 50 pg, at least 55 pg, at least 60 μg, at least 65 pg, at least 70 pg, at least 75 pg, at least 80pg, at least 85 pg, at least 90 pg, at least 95 pg, or at least 100 pg. The doses administered to a subject may number at least 1, 2, 3, 4, 5, 6, 7, 8, 9, 10, 11, or 12, or more. For antibodies or fragments thereof of the invention, the dosage administered to a patient may be 0.0001 mg/kg to 100 mg/kg of the patient's body weight. The dosage may be between 0,0001 mg/kg and 20 mg/kg, 0.0001 mg/kg and 10 mg/kg, 0.0001 mg/kg and 5 mg/kg, 0.0001 and 2 mg/kg, 0.0001 and 1 mg/kg, 0.0001 mg/kg and 0.75 mg/kg, 0.0001 mg/kg and 0.5 mg/kg, 0.0001 mg/kg to 0.25 mg/kg, 0.0001to 0.15 mg/kg, 0.0001 to 0.10 mg/kg, 0.001 to 0.5 mg/kg, 0.01 to 0.25 mg/kg or 0.01 to 0.10 mg/kg of the patient's body weight.

The dosage of the antibodies or fragments thereof of the invention may be calculated using the patient's weight in kilograms (kg) multiplied by the dose to be administered in mg/kg. The dosage of the antibodies or fragments thereof of the invention may be 150 μg/kg or less, 125 μg/kg or less, 100 μg/kg or less, 95 μg/kg or less, 90 μg/kg or less, 85 μg/kg or less, 80 μg/kg or less, 75 μg/kg or less, 70 μg/kg or less, 65 μg/kg or less, 60 μg/kg or less, 55 μg/kg or less, 50 μg/kg or less, 45 μg/kg or less, 40 μg/kg or less, 35 μg/kg or less, 30 μg/kg or less, 25 μg/kg or less, 20 μg/kg or less, 15 μg/kg or less, 10 μg/kg or less, 5 μg/kg or less, 2.5 μg/kg or less, 2 μg/kg or less, 1.5 μg/kg or less, 1 μg/kg or less, 0.5 μg/kg or less, or 0.5 μg/kg or less of a patient's body weight.

Unit dose of the antibodies or fragments thereof of the invention may be 0.1 mg to 20 mg, 0.1 mg to 15 mg, 0.1 mg to 12 mg, 0.1 mg to 10 mg, 0.1 mg to 8 mg, 0.1 mg to 7 mg, 0.1 mg to 5 mg, 0.1 to 2.5 mg, 0.25 mg to 60 mg, 0.25 mg to 40 mg, 0.25 mg to 20 mg, 0.25 to 15 mg, 0.25 to 12 mg, 0.25 to 10 mg, 0.25 to 8 mg, 0.25 mg to 7 mg, 0.25 mg to 5 mg, 0.5 mg to 2.5 mg, 1 mg to 20 mg, 1 mg to 15 mg, 1 mg to 12 mg, 1 mg to 10 mg, 1 mg to 8 mg, 1 mg to 7 mg, 1 mg to 5 mg, or 1 mg to 2.5 mg.

The dosage of the antibodies or fragments thereof of the invention may achieve a serum titer of at least 0.1 μg/ml, at least 0.5 μg/ml, at least 1 μg/ml, at least 2 μg/ml, at least 5 μg/ml, at least 6 μg/ml, at least 10 μg/ml, at least 15 μg/ml, at least 20 μg/ml, at least 25 μg/ml, at least 50 μg/ml, at least 100 μg/ml, at least 125 μg/ml, at least 150 μg/ml, at least 175 μg/ml, at least 200 μg/ml, at least 225 μg/ml, at least 250 μg/ml, at least 275 μg/ml, at least 300 μg/ml, at least 325 μg/ml, at least 350 μg/ml, at least 375 μg/ml, or at least 400 μg/ml in a subject. Alternatively, the dosage of the antibodies or fragments thereof of the invention may achieve a serum titer of at least 0.1 μg/ml, at least 0.5 μg/ml, at least 1 μg/ml, at least, 2 μg/ml, at least 5 μg/ml, at least 6 μg/ml, at least 10 μg/ml, at least 15 μg/ml, at least 20 μg/ml, at least 25 μg/ml, at least 50 μg/ml, at least 100 μg/ml, at least 125 μg/ml, at least 150 μg/ml, at least 175 μg/ml, at least 200 pg/ml, at least 225 pg/ml, at least 250 pg/ml at least 275 pg/ml, at least 300 pg/ml, at least 325 pg/ml, at least 350 pg/ml, at least 375 pg/ml, or at least 400 pg/ml in the subject.

Doses of antibodies or fragments thereof of the invention may be repeated and the administrations may be separated by at least 1 day, 2 days, 3 days, 5 days, 7 days, 10 days, 15 days, 30 days, 45 days, 2 months, 75 days, 3 months, or at least 6 months.

An effective amount for a particular patient may vary depending on factors such as the condition being treated, the overall health of the patient, the method route and dose of administration and the severity of side effects (see, e.g., Maynard et al., (1996) A Handbook of SOPs for Good Clinical Practice, Interpharm Press, Boca Raton, Fla.; Dent (2001) Good Laboratory and Good Clinical Practice, Urch PubL, London, UK).

The route of administration may be by, e.g., topical or cutaneous application, injection or infusion by intravenous, intraperitoneal, intracerebral, intramuscular, intraocular, intraarterial, intracerebrospinal, intralesional, or by sustained release systems or an implant. See for example Sidman et al., (1983) Biopolymers 22:547-556; Langer et al. (1981) J. Biomed. Mater. Res. 15:167-277; Langer (1982) Chem. Tech. 12:98-105; Epstein et al, (1985) Proc. Natl. Acad. Sci, USA 82:3688-3692; Hwang et at. (1980) Proc. Natl. Acad. Sci, USA 77:4030-4034; U.S. Pat. Nos. 6,350,466 and 6,316,024). Where necessary, the composition may also include a solubilizing agent and a local anesthetic such as lidocaine to ease pain at the site of the injection. In addition, pulmonary administration can also be employed, e.g., by use of an inhaler or nebulizer, and formulation with an aerosolizing agent. See for example U.S. Pat. Nos. 6,019,968, 5,985,320, 5,985,309, 5,934,272, 5,874,064, 5,855,913, 5,290,540, and 4,880,078; and PCT Publication Nos. WO 92/19244, WO 97/32572, WO97/44013, WO 98/31346, and WO 99/66903, or any similar reference known to those of skill in the art.

A composition of the present invention may also be administered via one or more routes of administration using one or more of a variety of methods known in the art. As will be appreciated by the skilled artisan, the route and/or mode of administration will vary depending upon the desired results. Selected routes of administration for antibodies or fragments thereof of the invention include intravenous, intramuscular, intradermal, intraperitoneal, subcutaneous, spinal or other parenteral routes of administration, for example by injection or infusion. Parenteral administration may represent modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, a composition of the invention can be administered via a non-parenteral route, such as a topical, epidermal or mucosal route of administration, for example, intranasally, orally, vaginally, rectally, sublingually or topically. In one embodiment, the antibodies or fragments thereof of the invention is administered by infusion.

In another embodiment, the multi-specific epitope binding protein of the invention is administered subcutaneously. If the antibodies or fragments thereof of the invention are administered in a controlled release or sustained release system, a pump may be used to achieve controlled or sustained release. See for example Langer et al., supra; Sefton, (1987) CRC Crit. Ref Biomed. Eng. 14:20; Buchwald et al., (1980), Surgery 88:507; Saudek et al, (1989) N. Engl. J. Med. 321: 574).

Polymeric materials can be used to achieve controlled or sustained release of the therapies of the invention. See for example Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas. (1983) J. Macromol. Sci. Rev. Macromol. Chem. 23:61: see also Levy et al., (1985) Science 228:190; During et al, (1989) Ann. Neurol. 25:351; Howard et al, (1989) J. Neurosurg. 7 1:105): U.S. Pat. Nos. 5,679,377; 5,916,597; 5,912,015; 5,989,463; 5,128,326; WO 99/15154; and WO 99/20253. Examples of polymers used in sustained release formulations include, but are not limited to, poly (2-hydroxy ethyl methacrylate), poly (methyl methacrylate), poly (acrylic acid), poly (ethylene-co-vinyl acetate), poly (methacrylic acid), polyglycolides (PLG), polyanhydrides, poly (N-vinyl pyrrolidone), poly (vinyl alcohol), polyacrylamide, poly (ethylene glycol), polylactides (PLA), poly (lactide-co-glycolides) (PLGA), and polyorthoesters. In one embodiment, the polymer used in a sustained release formulation is inert, free of leachable impurities, stable on storage, sterile, and biodegradable. A controlled or sustained release system can be placed in proximity of the prophylactic or therapeutic target, thus requiring only a fraction of the systemic dose. See for example Goodson, in Medical Applications of Controlled Release, supra, vol. 2, pp. 115-138 (1984)).

Controlled release systems are discussed in the review by Langer, (1990), Science 249: 1527-1533). Any technique known to one of skill in the art can be used to produce sustained release formulations comprising one or more antibodies or fragments thereof of the invention. See for example, U.S. Pat. No. 4,526,938, WO 91/05548, WO 96/20698, Ning et al, (1996), Radiotherapy & Oncology 39:179-189, Song et al, (1995) PDA Journal of Pharmaceutical Science & Technology 50:372-397, Cleek et al., (1997) Pro. Intl Symp. Control. Rel. Bioact. Mater. 24:853-854, and Lam et al. (1997) Proc. Intl Symp. Control Rel. Bioact. Mater. 24:759-760.

If the antibodies or fragments thereof of the invention are administered topically, they can be formulated in the form of an ointment, cream, transdermal patch, lotion, gel, shampoo, spray, aerosol, solution, emulsion, or other form well-known to one of skill in the art. See for example Remington's Pharmaceutical Sciences and Introduction to Pharmaceutical Dosage Forms, 19th ed., Mack Pub. Co., Easton, Pa. (1995). For non-sprayable topical dosage forms, viscous to semi-solid or solid forms comprising a carrier or one or more excipients compatible with topical application and having a dynamic viscosity, in some instances, greater than water are typically employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations wherein the active ingredient, in some instances, in combination with a solid or liquid inert carrier, is packaged in a mixture with a pressurized volatile (e.g., a gaseous propellant, such as freon) or in a squeeze bottle. Moisturizers or humectants can also be added to pharmaceutical compositions and dosage forms if desired. Examples of such additional ingredients are well-known in the art.

If the compositions comprising antibodies or fragments thereof are administered intranasally, it can be formulated in an aerosol form, spray, mist or in the form of drops. In particular, prophylactic or therapeutic agents for use according to the present invention can be conveniently delivered in the form of an aerosol spray presentation from pressurized packs or a nebulizer, with the use of a suitable propellant (e.g., dichlorodifluoromethane, trichlorofluoromethane, dichlorotetrafluoroethane, carbon dioxide or other suitable gas). In the case of a pressurized aerosol the dosage unit may be determined by providing a valve to deliver a metered amount. Capsules and cartridges (composed of, e.g., gelatin) for use in an inhaler or insufflator may be formulated containing a powder mix of the compound and a suitable powder base such as lactose or starch.

Methods for co-administration or treatment with a second therapeutic agent are known in the art. An effective amount of therapeutic may decrease the symptoms by at least 10%: by at least 20%; at least about 30%>; at least 40%>, or at least 50%. Additional therapies (e.g., prophylactic or therapeutic agents), which can be administered in combination with the antibodies or fragments thereof of the invention may be administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours apart from the antibodies or fragments thereof of the invention. The two or more therapies may be administered within one same patient visit.

The antibodies or fragments thereof of the invention and the other therapies may be cyclically administered. Cycling therapy involves the administration of a first therapy (e.g, a first prophylactic or therapeutic agent) for a period of time, followed by the administration of a second therapy (e.g., a second prophylactic or therapeutic agent) for a period of time, optionally, followed by the administration of a third therapy (e.g., prophylactic or therapeutic agent) for a period of time and so forth, and repeating this sequential administration, i.e., the cycle in order to reduce the development of resistance to one of the therapies, to avoid or reduce the side effects of one of the therapies, and/or to improve the efficacy of the therapies.

In certain embodiments, the antibodies or fragments thereof of the invention can be formulated to ensure proper distribution in vivo. For example, the blood-brain barrier (BBB) excludes many highly hydrophilic compounds. To ensure that the therapeutic compounds of the invention cross the BBB (if desired), they can be formulated, for example, in liposomes. For methods of manufacturing liposomes. See for example, U.S. Pat. Nos. 4,522,811; 5,374,548; and 5,399,331. The liposomes may comprise one or more moieties which are selectively transported into specific cells or organs, thus enhance targeted drug delivery (see, e.g., Ranade, (1989) J. Olin. Pharmacol. 29:685).

The invention provides protocols for the administration of pharmaceutical composition comprising antibodies or fragments thereof of the invention alone or in combination with other therapies to a subject in need thereof. The therapies (e.g., prophylactic or therapeutic agents) of the combination therapies of the present invention can be administered concomitantly or sequentially to a subject. The therapy (e.g., prophylactic or therapeutic agents) of the combination therapies of the present invention can also be cyclically administered. Cycling therapy involves the administration of a first therapy (e.g., a first prophylactic or therapeutic agent) for a period of time, followed by the administration of a second therapy (e.g., a second prophylactic or therapeutic agent) for a period of time and repeating this sequential administration, i.e., the cycle, in order to reduce the development of resistance to one of the therapies (e.g., agents) to avoid or reduce the side effects of one of the therapies (e.g., agents), and/or to improve, the efficacy of the therapies.

The therapies (e.g., prophylactic or therapeutic agents) of the combination therapies of the invention can be administered to a subject concurrently. The term “concurrently” is not limited to the administration of therapies (e.g., prophylactic or therapeutic agents) at exactly the same time, but rather it is meant that a pharmaceutical composition comprising antibodies or fragments thereof of the invention are administered to a subject in a sequence and within a time interval such that the antibodies of the invention can act together with the other therapies to provide an increased benefit than if they were administered otherwise. For example, each therapy may be administered to a subject at the same time or sequentially in any order at different points in time; however, if not administered at the same time, they should be administered sufficiently close in time so as to provide the desired therapeutic or prophylactic effect. Each therapy can be administered to a subject separately, in any appropriate form and by any suitable route.

In various embodiments, the therapies (e.g., prophylactic or therapeutic agents) are administered to a subject less than 15 minutes, less than 30 minutes, less than 1 hour apart, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, 24 hours apart, 48 hours apart, 72 hours apart, or 1 week apart. In other embodiments, two or more therapies (e.g., prophylactic or therapeutic agents) are administered to a within the same patient visit.

The prophylactic or therapeutic agents of the combination therapies can be administered to a subject in the same pharmaceutical composition. Alternatively, the prophylactic or therapeutic agents of the combination therapies can be administered concurrently to a subject in separate pharmaceutical compositions. The prophylactic or therapeutic agents may be administered to a subject by the same or different routes of administration. The invention having been fully described, it is further illustrated by the following examples and claims, which are illustrative and are not meant to be further limiting.

Throughout this disclosure, various quantities, such as amounts, sizes, dimensions, proportions and the like, are presented in a range format. It should be understood that the description of a quantity in range format is merely for convenience and brevity and should not be construed as an inflexible limitation on the scope of any embodiment. Accordingly, the description of a range should be considered to have specifically disclosed all the possible subranges as well as all individual numerical values within that range unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 4.62, 5, and 5.9. This applies regardless of the breadth of the range. The upper and lower limits of these intervening ranges may independently be included in the smaller ranges, and are also encompassed within the disclosure, subject to any specifically excluded limit in the stated range. Where the stated range includes one or both of the limits, ranges excluding either or both of those included limits are also included in the disclosure, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “includes,” “comprises”, “including” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Additionally, it should be appreciated that items included in a list in the form of “at least one of A, B, and C” can mean (A); (B); (C); (A and B): (B and C); (A and C): or (A, B, and C). Similarly, items listed in the form of “at least one of A, B, or C” can mean (A); (B); (C); (A and B); (B and C); (A and C); or (A, B, and C).

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

### General Materials and Methods

### Human Samples

A total of 25 human sera and matching urine samples were obtained from diabetic nephropathy (DN) patients seen at the Renal Associates, MGH by Dr. Kristin M. Corapi under MGH IRB protocol #2014P001943 in the period of 2014-2018. In addition, in the period between 2018 and 2020, we obtained 57 sera from the Mass General Brigham Biobank. Forty patients were diagnosed with DN and were not receiving insulin therapy when serum samples were collected. Forty-two patients were diagnosed with DN and were receiving insulin therapy when the serum samples were collected. Statistical analysis was performed using SPSS 27 (IBM, Armony NY). The inventors compared the prevalence of hD2D3 (reported as a %) between patients with DN on or not on insulin using the chi-square test. To assess whether hD2D3 was independently associated with being on insulin, the inventors used a logistic regression model with insulin therapy as the dependent categorical variable and presence of hD2D3, hemoglobin A1c % and suPAR (log-transformed base 2) as independent variables. Lastly, the inventors computed the area under the curve (AUC) for hD2D3 and suPAR in separate models and combined to assess their ability to differentiate between those on or not on insulin therapy. AUCs were compared using the Delong test. A two-sided P-value<0.05 was adopted to indicate statistical significance.

### Reagents

RPMI 1640 medium (11875-093), CMRL medium (21540-026): FBS (10082-147), penicillin-streptomycin (15140-122), antibiotic/antimycotic (15240-096) (penicillin, streptomycin and Amphtericin B) were from Gibco. EZ-link Micro Sulfo-NHS-Biotinylation Kit (21925), Zeba™ Spin Desalting Column, and Pierce™ IP RIPA Buffer (89901) were from Thermo Fisher Scientific. β-mercaptoethanol (M6250) and ITS (insulin-transferrin-sodium selenite) media supplement (13146) were from Sigma-Aldrich. The reagents used for in vitro GSIS (MIN6 cells, mouse islets, and human islets) were from Sigma-Aldrich. Sodium pyruvate (Corning, 10-013-CV); protease inhibitor cocktail tablet (Roche. 11836170001); Chymotrypsin (Roche, 11 418 467001): uPAR (R&D systems, 807-UK/CF); streptavidin Mag Sepharose beads (GE Healthcare, 28-9857-99); N-Glycanase (PROzyme, GKE-5006A).

### Cell Culture

Mouse MIN6 cells (a gift from Dr. Decheng Ren, University of Chicago) were grown as described before. See Ren et al., “Role of BH3-only molecules Bim and Puma in beta-cell death in Pdx1 deficiency,” (2014) Diabetes 63: pp. 2744-2750. Immortalized human podocytes were cultured according to published protocol (Saleem et al., 2002). Mouse islets were cultured in RPMI 1640 medium containing 10% FBS, 1% penicillin/streptomycin, and 50 μM β-mercaptoethanol. Human islets were cultured in CMRL medium containing 10% FBS and 1% penicillin/streptomycin.

### Antibodies

Rabbit anti-c-Myc antibody (Sigma-Aldrich, PLA0001), guinea pig anti-insulin antibody (Abcam, ab7842), mouse anti-glucagon antibody (Sigma-Aldrich, G2654). Secondary antibody for insulin labeling was Alexa Fluor 488-conjugated goat anti-guinea pig IgG (Invitrogen) and for glucagon labeling was Alexa Fluor 594-conjugated chicken anti-rabbit IgG (Invitrogen). uPAR (R4)-BSA Free (Novusbio, NBP2-41379); Rabbit anti-uPAR (Bethyl, A304-462A); Mouse uPAR polyclonal antibody (R&D systems, AF534); anti-goat IgG HRP antibody (Thermo Fisher, HAF109); anti-rabbit IgG HRP antibody (Thermo Fisher, G-21234); AP5 antibody (Blood Center of Wisconsin); paxillin antibody Y-113 (Abcam, ab32081).

### Standard Procedures

Mouse pancreatic islets isolation was performed as described. See Zhu et al., “Kindlin-2 modulates MafA and beta-catenin expression to regulate beta-cell function and mass in mice.” (2020) Nat Commun 11: p. 484. Islets were picked up manually under a dissecting microscope (Nikon Instruments Inc, Melville, NY, USA). Pancreatic β-cell area and β-cell mass were calculated as described. Id. AP5 assay using human podocytes was performed as described. See Hayek et al., “A tripartite complex of suPAR, APOLI risk variants and alphavbeta3 integrin on podocytes mediates chronic kidney disease,” (2017) Nat Med 23: pp. 945-953.

### Immunoprecipitation-Coupled to Western Blot Analysis (IP-WB)

Human sera and urine samples: Human sera or urine samples were diluted (1:1) with RIPA buffer containing protease inhibitor cocktail tablet (RIPA-PI) and precleared using Streptavidin Mag Sepharose beads. Human uPAR (R4) antibody (Novusbio, NBP2-41379) was biotinylated using EZ-link Micro Sulfo-NHS-Biotinylation Kit. Biotinylated uPAR (R4) antibody was added to the precleared samples. Subsequently, Streptavidin Mag Sepharose beads were added to the rotating samples. The total immunoprecipitation (IP) time for generating samples for mass spectrometric analysis was 24 hours, and for IP-WP was 3-4 hours. The magnetic beads were washed with RIPA buffer and the bound fraction was deglycosylated using N-Glycanase. The proteins were analyzed using SDS-PAGE. Western blot analysis was performed using polyclonal rabbit anti-uPAR (Bethyl, A304-462A). Recombinant human uPAR (R&D systems, 807-UK/CF). chymotrypsin digested uPAR, or recombinant hD2D3 were used as positive controls. For mass spectrometric analysis, proteins immunoprecipitated from patient sera or urine were deglycosylved and subjected to SDS PAGE and the protein bands were excised from the gel and submitted to the Taplin Biological Mass Spectrometry Facility at Harvard Medical School.

Mouse sera: Mouse sera were diluted, precleared and immunoprecipitated as explained for human sera above. The antibody used to IP mouse proteins was the polyclonal mouse uPAR antibody (R&D systems, AF534). Recombinant mouse suPAR and recombinant mouse D2D3 were used as positive controls. Western blot was performed using rabbit anti-c-myc antibody (Sigma-Aldrich, PLA0001) and rabbit IgG-HRP antibody

### β3 Integrin Activation Assay Using Human Podocytes

The experiments were performed as described elsewhere. See Hayek et al. 2017. Where indicated, the cells were treated with serum-free media=hD2D3 for one set, and HS±hD2D3 for another set. Images were acquired using a Zeiss microscope. The intensity of AP5 or paxillin was quantified using Fiji, ImageJ (NIH). Non-specific nuclear staining was disregarded while performing quantitative analysis. Data are represented as a ratio of AP5/paxillin intensity relative to the control. An unpaired two-tailed t-test was performed using Prism (GraphPad) to compare the treatments.

### Cloning, Expression and Purification of Recombinant Proteins

Gene encoding mouse uPAR isoform 1 (GenBank NM_011113.4) was amplified using total RNA isolated from cultured mouse podocytes using known forward and reverse primers. The PCR products were digested with the restriction enzymes HindIII and EcoRI, and subcloned into the pSec Tag2A vector containing C-terminal Myc/His tag (Thermo Fisher. V90020). pSecTag2A-suPAR/D2D3 plasmids were transiently transfected into the FreeStyle™ 293-F cells (Thermo Fisher, 12347-019). Recombinant proteins were purified from the culture medium using Pierce anti-c-Myc agarose (Thermo Fisher. 20168) based on the manufacturer's protocol.

### Mice

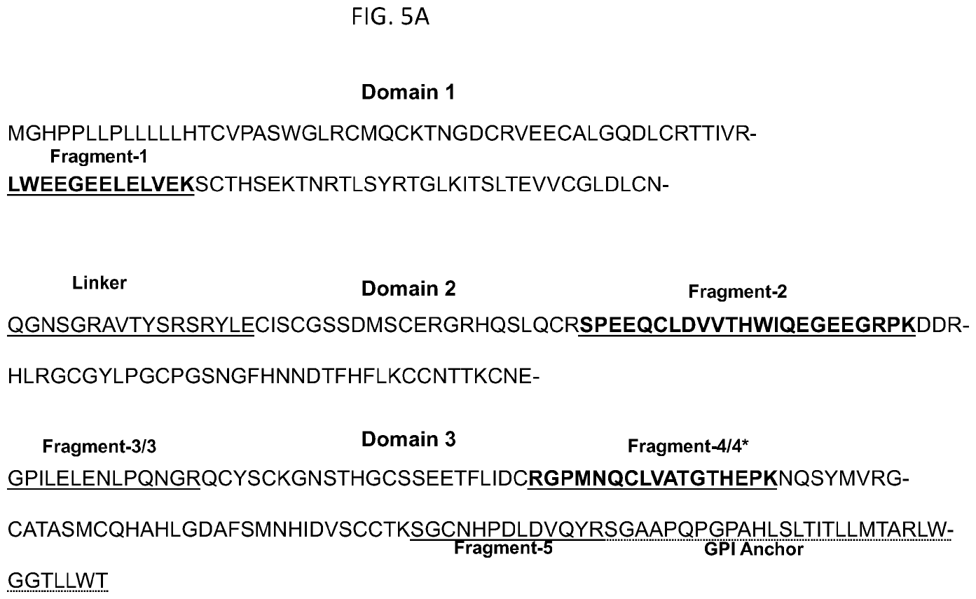

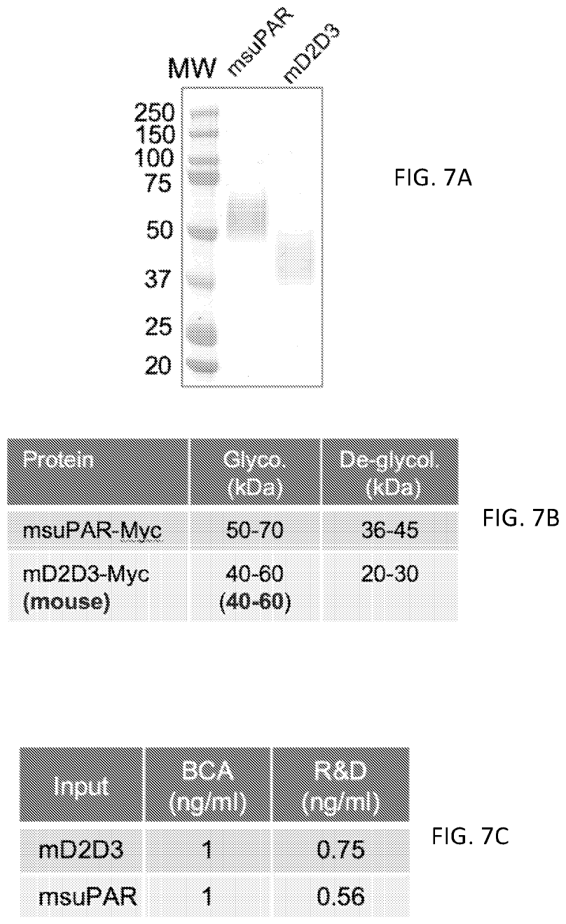

Mice expressing full length suPAR (suPAR-Tg) was previously described. See Hahm et al. 2017. Mice expressing the D2D3 protein (D2D3-Tg) was generated at the Transgene Facility, University of Miami. DNA encoding C-terminal D2D3 protein of mouse suPAR isoform 1 (uniprot.org/uniprot/P35456), corresponding to NM_011113.4 in GenBank and covering amino acids 117-298 of the mature was placed under control of the aP2 promoter cassette (Wang et al., 2010) to achieve adipocyte-specific expression of the fragment. In addition, DNA encoding the secretion signal peptide (Igk) was placed at the N-terminal alanine 117 of D2D3 protein to ensure that the fragment is secreted into the circulation. The signal peptide is post-translationally cleaved between glycine (G) and aspartic acid (D, underlined). Furthermore, DNA encoding GPI-anchor was replaced with DNA encoding a Myc-tag (see below, FIG. 5A and FIG. 7A-C).

Genotyping-positive founder mice (D2D3-Tg) were backcrossed to C57BL/6 mice for at least 5 generations to establish the colony. D2D3-Tg mice were viable and fertile. Mice were maintained on either a regular diet or high-fat diet (HFD). All animal experiments were carried out according to the NIH Guide for Care and Use of Experimental Animals and approved by the Rush University Institutional Animal Care and Use Committee (IACUC) protocol #19-014.

Real-Time RT-PCR (qPCR)

RNA was isolated with TRIzol (Thermo Fisher, 15596-026) and cDNA was generated using high-capacity cDNA reverse transcription kit (Thermo Fisher, 4368814). qPCR was performed using SsoAdvanced Universal SYBR Green Supermix (BioRad, 172-5271). All PCR data were normalized by the Gapdh gene expression level. qPCR primer sequences to identify D2D3 (including part of the Myc tag) were utilized.

### Immunohistochemical Staining

Paraffin-embedded fat or pancreas sections (5 μm thick) were deparaffinized in xylene and rehydrated using graded ethanol series (100%, 95%, 70%), followed by rinse in distilled water. Antigen retrieval was carried out by boiling the slides in a microwave in sodium citrate solution (pH 6.0). After incubation with a blocking solution (2% BSA. 0.3% Triton X-100 in PBS) for 1 hour at room temperature. the sections were stained with anti-c-Myc antibody for detection of D2D3 protein (1:2000), anti-insulin antibody (1:500), or anti-glucagon antibody (1:300) followed by secondary antibody. Images were acquired using LSM 700 confocal microscope (Carl Zeiss).

### Cell Proliferation and Apoptosis

Pancreatic sections from each group of mice were stained with antibodies against insulin or Ki67 as previously described. See Zhu et al. 2020. For quantitative analysis, ImageJ (NIH) was used to count the number of DAPI positive cells within the insulin staining area. At least 1000 insulin-positive cells per mouse were counted and Ki67-positive cells were determined. Ki67-positive cells were normalized to total insulin-positive cells in the same area. Cell survival was evaluated using the In Situ Cell Death Detection Kit (Roche Applied Sciences, 11684795910) following the instructions from the manufacturer. Briefly. after dewaxation, antigen retrieval was performed using proteinase K (10) μg/ml in 10 mM Tris/HCl, pH 74) at room temperature for 15 min. The slides were washed twice with 1×PBS and incubated in TUNEL reaction mixture (50 μl of Enzyme solution plus 450 μl Label Solution) for 60 min at 37° C. The pancreas sections were further stained with an antibody insulin and DAPI. The sections were examined using an LSM 700 laser scanning fluorescence confocal microscope running ZEN software (Zeiss). TUNEL-positive cells were normalized to total insulin-positive cells in the same area as what we did for Ki67 quantification.

### Assessment of Renal Function and Insulin Levels

Urinary albumin was measured using mouse albumin ELISA (Bethyl Labs, E99-134), and creatinine was measured using an enzymatic assay kit (Cayman Chemical, 500701). Albumin-to-creatinine (ACR) ration was calculated. Kidney function was determined by measuring blood urea nitrogen level (BUN) using BioAssay Systems (DIUR-500) and serum creatinine levels were determined using Crystal Chem assay (80350). Pancreas function was determined by measuring levels of C-peptide using mouse C-peptide ELISA kit (90050) and insulin levels using ultra-sensitive mouse insulin ELISA kit (90080) from Crystal Chem. Levels of IL-6 were measured by R&D Systems (M6000B) and CRP were measured using Crystal Chem assay (80350).

In vivo Glucose Tolerance Test (GTT) and Glucose Stimulated Insulin Secretion GSIS Assays

For both assays, animals fasted overnight. Glucose was administered by intraperitoneal injection at concentration of 2 g/kg body weight. Blood samples were taken at indicated times via tail nick as the indicated time points. Levels of blood glucose and serum insulin were measured using a glucose meter (Bayer HealthCare) and ELISA assay (Crystal Chem, 90080), respectively.

In vitro GSIS Assays

GSIS was performed on MIN6 cells and isolated mouse or human islets. MIN6 cells were maintained in the culture medium containing 1 g/L D-glucose. Indicated concentrations of recombinant mD2D3 or mouse suPAR were added to the cells for 24 hours. Before initiation of GSIS, cells were washed twice with KRBH (137 mM NaCl, 4.7 mM KCl, 1.2 mM KH2PO4, 1.2 mM MgSO4, 2.5 mM CaCl2-2H2O, 25 mM NaHCO3, 10 mM HEPES) supplemented with 0.2% BSA and 1 g/L (5.5 mM) D-glucose. Cells were placed in a low glucose KRBH media (5.5 mM glucose, 0.2% BSA) for 30 min at 37° C., followed by KRBH (0.2% BSA) media containing either 5.5 mM or 20 mM glucose for one hour at 37° C. For mouse islets. after overnight culture in 5.5 mM RPMI, size-matched islets were treated with BSA. IgG, mD2D3 or suPAR protein as indicated in the figure legends. Islets were then placed into KRBH buffer containing 2.8 mM glucose for one hour and subjected to 2.8 mM and 16.7 mM glucose stimulation. For human islets, which were obtained from the UVA Islet Microfluidic Laboratory of the University of Virginia or ADI IsletCore Alberta Diabetes Institute of the University of Alberta (Edmonton, Canada), after overnight culture in CMRL medium, islets were incubated in 10% of human serum for 4 days.

When indicated, 10 ng/ml of human recombinant suPAR or hD2D3 protein was added to human sera. GSIS was initiated by the end of the 4th day as described for mice islets above. Released insulin in supernatants were determined using mouse insulin ELISA kit (Crystal Chem, 90080) or human insulin ELISA kit (Crystal Chem, 90095). The total protein was extracted by sonication for 2 minutes in 500 μl acid/ethanol (0.18 M HCl in 95% ethanol) solution as previously described. See Zhu et al. 2020.

The study protocol regarding the work with human islets was approved by the Institutional Review Board of Rush University Medical Center (IRB protocol #14051401-IRB01-AM07).

### Antibody Treatment

2-month-old D2D3-Tg mice were treated twice a week with 0.5 mg kg-1 body weight of mouse uPAR antibody (R&D Systems, AF534), intraperitoneally injected, for four weeks.

### Extracellular Flux Analysis

Experiments were performed as described in (Altintas et al., 2021). See Altintas et al., “Metabolic Changes in Peripheral Blood Mononuclear Cells Isolated From Patients With End Stage Renal Disease,” (2021) Front Endocrinol (Lausanne) 12:629239. Cellular oxygen consumption rates (OCR) and extracellular acidification rates (ECAR) were detected by Seahorse XFe24 Analyzer (Agilent Technologies, Santa Clara, CA) using the Cell Mito Stress Test and Glycolysis Stress Test kits (both from Agilent), respectively. Mouse insulinoma 6 (MIN6) cells (6×104) were seeded onto each well of a 24-well assay plate (Agilent) and allowed to attach overnight and grow for 24 h in the regular culture medium. Then, the medium was replaced with the low-glucose (2.8 mM) culture medium and cells were treated with 10 and 100 ng/ml D2D3 protein of mouse D2D3 protein for 24 hours. On the day of the assay, the medium was replaced with the bicarbonate-free XF DMEM assay medium, pH 7.4 (Agilent) supplemented with 1 mM sodium pyruvate, 2 mM glutamine, and 2.8 mM glucose to optimize the respiration condition. The plate was incubated in a non-CO2 incubator for 30 to 60 min before being transferred to the analyzer. For the mitochondrial respiration analysis, the first three measurements of OCR were recorded under basal conditions. To stimulate the cellular oxygen consumption, 175 mM glucose solution was injected (final concentration in each well: 20 mM) and OCR was measured for another 60 min. This was followed by the sequential injections of 10× concentrations oligomycin (complex V inhibitor: final concentration in each well: 1.0 μM), FCCP (uncoupler: 1.25 μM), rotenone and antimycin A (inhibitors of complex I and complex III, respectively: 0.5 μM, each). Three readings of OCR were recorded after each injection and all recordings were 8 minutes apart. This protocol allowed for an estimation of the basal respiration, proton leak, ATP production, maximal respiration, spare respiratory capacity and non-mitochondrial respiration rates as explained previously. Id. For the glycolysis assay, XF DMEM assay medium was supplemented with only 2 mM glutamine whereas glucose (10 mM), oligomycin (1 μM) and 2-deoxy-glucose (2DG: 50 mM) were sequentially injected after basal ECAR readings. Three readings of ECAR were recorded after each injection and all recordings were 8 minutes apart. Basal acidification (ECAR prior to glucose injection), glycolysis (difference between ECAR before and after addition of glucose) and non-glycolytic acidification (residual ECAR after addition of oligomycin and 2DG) rates were computed to characterize glycolysis. Extracellular flux readings were normalized to protein content in each well. Therefore, OCR and ECAR measures are represented as pmol/min/μg protein and mpH/min/mg protein, respectively.

### Transmission Electron Microscopy (TEM) of Kidney Samples

Mouse renal tissues were cut into 2×2 mm pieces. The tissue was fixed in Trump's fixative (EMS, 11750), dehydrated with graded ethanol, dried using a 850 Critical Point Dryer (EMS), gold-coated on a Cressington 108 Auto Sputter Coater (Ted Pella), and post fixed in 1% OsO4 for 1 hour on ice. Then, the tissue was washed in 0.1 M cacodylate buffer, dehydrated, and embedded in Epon812 (EMS). Ultrathin (70 nm) sections were collected onto Formvar-coated Ni slot grids (EMS) and stained for 15 min in 5% uranyl acetate and 0.1% lead citrate. Electron micrographs were taken with Sigma HDVP Electron Microscope (Zeiss).

### Platinum Replica Electron Microscopy (PR-EM) and Ultrathin Sections Electron Microscopy (UTS-EM) on MIN6 Cells

MIN6 cells were grown on poly-L-lysine (Sigma, P4707) coated glass coverslips for PR-EM or ACLAR plastic sheets for UTS-EM. GSIS was performed as described above. For PR-EM, cells were detergent extracted and processed as described. See Svitkina “Imaging Cytoskeleton Components by Electron Microscopy,” (2016) Methods Mol Biol 1365: pp. 99-118. Samples were dehydrated in a critical point dryer Autosamdri-815 (Tousimis) and coated with a 2 nm layer of platinum and stabilized with 5 nm of carbon using EM ACE600 sputter coater (Leica). The platinum-carbon replica was released from the glass coverslips on 10% hydrofluoric acid and picked up on to EM grids. For UTS-EM, cells were detergent-extracted and fixed with 2.5% glutaraldehyde. 1.25% paraformaldehyde and 0.03% picric acid in 0.1 M sodium cacodylate buffer, pH 7.4 for 1 hour at room temperature. Then, cells were rinsed in in 0.1 M sodium cacodylate buffer three times, followed by post-fixation with 1% osmium tetroxide, OsO4 and 1.5% potassium ferrocyanide, K3(Fe(CN)6) for 1 hour at room temperature. Samples were dehydrated the same as for PR-EM, subsequently embedded in TAAB Epon (Marivac Canada Inc) and polymerized at 60° C. for 48 hours. After polymerization the ACLAR was peeled off and ultrathin sections (about 50-70 nm) were cut on a Reichert ultracut S microtome (Leica), picked up on to EM grids and stained with lead citrate. Samples for PR-EM and UTS-EM were examined using a JEM 1011 model TEM (JEOL) at an acceleration voltage of 80 kV. Images were captured by CCD camera (Gatan) and presented in inverted contrast. The vesicles and membrane were colored using the brush tool with 50% opacity in Photoshop. The vesicle size was measured using the ruler tool in ImageJ software (NIH).

Further reference is made to the following experimental examples.

### EXAMPLES

The following examples are provided for the purpose of illustrating various embodiments of the invention and are not meant to limit the present disclosure in any fashion. The present examples, along with the methods described herein are presently representative of preferred embodiments, are provided only as examples, and are not intended as limitations on the scope of the invention. Changes therein and other uses which are encompassed within the spirit of the disclosure as defined by the scope of the claims will occur to those skilled in the art.

### Example 1

### D2D3 in Patients With Diabetic Neuropathy (DN)

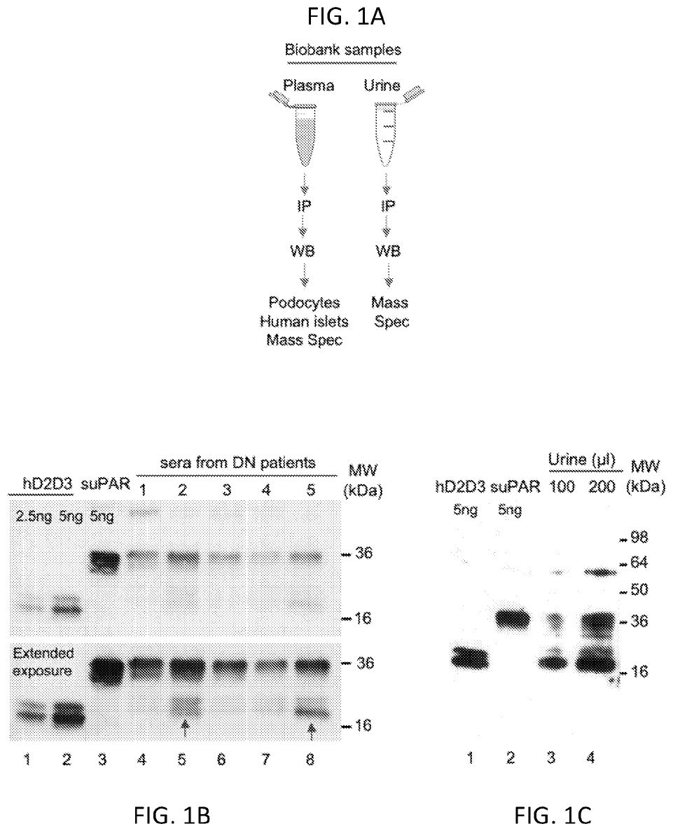

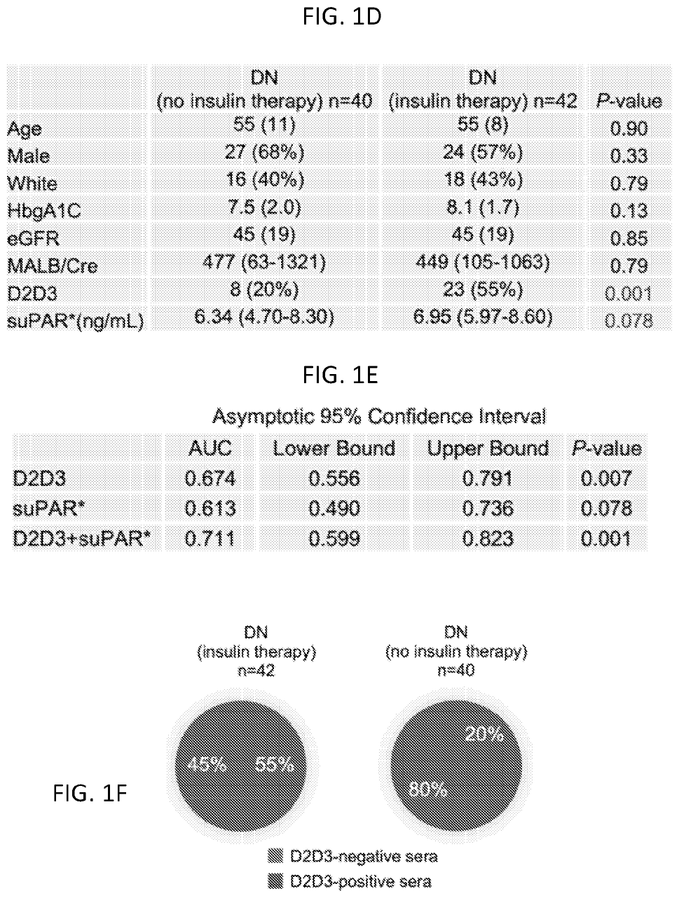

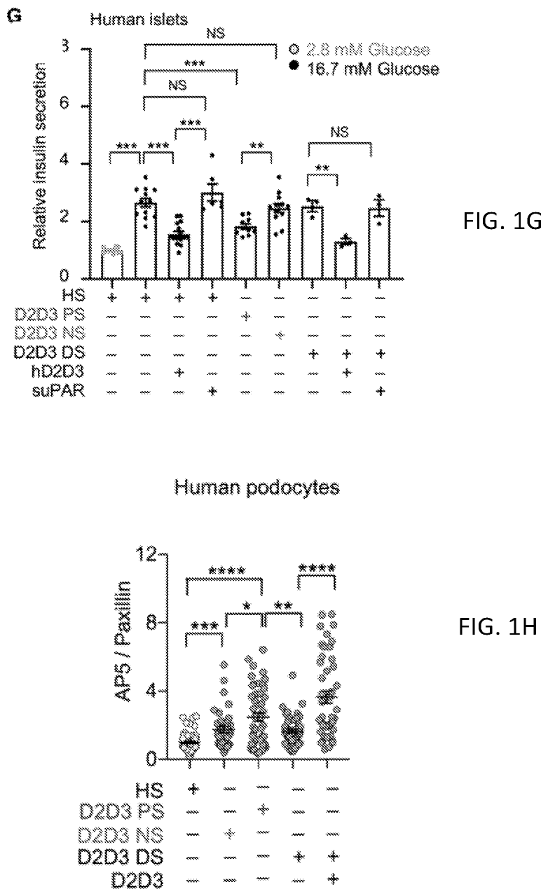

Increased expression of β3 integrin has been associated with DN. See Wilson et al., “The single-cell transcriptomic landscape of early human diabetic nephropathy,” Proc Natl Acad Sci USA 116: pp. 19619-19625 (2019). Thus, the inventors first sought to determine whether patients with DN have circulating D2D3 proteins (FIG. 1A).

FIG. 1A-H depicts that the D2D3 protein discriminates between DN patients on insulin therapy and those that are not. (FIG. 1A) Schematic of human sample analyses. (FIG. 1B and FIG. 1C) suPAR and D2D3 protein detection by IP-WB in DN patient sera (FIG. 1B) or urine (FIG. 1C). Controls: recombinant human suPAR or chymotrypsin digested-suPAR representing D2D3 protein. (FIG. 1D) Study patients' characteristics. (FIG. 1E) Comparison (area under the curve, (AUC) values) between patients who were or were not on insulin therapy. (FIG. 1F) Pie chart showing the distribution (%) of D2D3-positive and D2D3-negative sera in DN patients who were or were not on insulin therapy. (FIG. 1G) Scatter dot plots representing levels of GSIS from human islets in the presence of human sera (n=3 experiments). Healthy donor islets were incubated in 10% healthy sera (HS), D2D3-positive sera (D2D3 PS, pooled from 8 patients), D2D3-negative sera (D2D3 NS, pooled from 6 patients), or D2D3 PS that were immunodepleted using an anti-uPAR antibody (R4) (D2D3 DS). When indicated, 10 ng/ml of recombinant hD2D3 or suPAR were added. (FIG. 1 FIG. 1H) Scatter dot plots showing activation of b3 integrin on human podocytes (cells>35) treated with sera as described in (G), excluding hD2D3 concentration of 2.5 ng/ml. Immunofluorescence analysis was performed using an anti-paxillin antibody (focal adhesions), and an AP5 antibody (activated b3 integrin). FIG. 1G and FIG. 1H, error bar, mean±SEM (*P<0.05, **P<0.01, ***P<0.001, ****P<0.0001, unpaired/-test), ns, not significant.

FIG. 5A,B shows suPAR-specific peptides in human samples. (FIG. 5A) Amino acid sequence of human uPAR protein isoform 1 (SEQ ID NO: 1). Distinct domains are labeled, Domain 1 (D1), Domain 2 (D2), and Domain 3 (D3). Linker between D1 and D2, and the GPI anchor underlined and labeled. Bold letters demarcate suPAR-specific peptides detected by mass spectrometry, the details of which are shown herein. Specifically shown are Fragments-1 to Fragment-5. * Additional fragment generated within the Fragment-4. Fragment-3 spans over D2 and D3 domain and thus is marked 3/3. (FIG. 5B) Coomassie gel showing proteins immunoprecipitated from sera using anti-uPAR antibody (R4). Serum samples (10 ml) from 14 DN patients that also had a D2D3-like fragment in their sera were used in the experiment. The protein band indicated in the figure was cut out of the gel and analyzed using mass spectrometry.

Human uPAR Protein and Target Fragments

Human uPAR Protein Isoform 1:
MGHPPLLPLLLLLHTCVPASWGLRCMQCKTNGDCRVEECALGQDLCRTTIVR
LWEEGEELELVEKSCTHSEKTNRTLSYRTGLKITSLTEVVCGLDLCNQGNSGRAVTYSRS
RYLECISCGSSDMSCERGRHQSLQCRSPEEQCLDVVTHWIQEGEEGRPKDDRHLRGCGY
LPGCPGSNGFHNNDTFHFLKCCNTTKCNEGPILELENLPQNGRQCYSCKGNSTHGCSSEE
TFLIDCRGPMNQCLVATGTHEPKNQSYMVRGCATASMCQHAHLGDAFSMNHIDVSCCT
KSGCNHPDLDVQYRSGAAPQPGPAHLSLTITLLMTARLWGGTLLWT (SEQ ID NO: 1)
uPAR Domain 1 (D1):
MGHPPLLPLLLLLHTCVPASWGLRCMQCKTNGDCRVEECALGQDLCRTTIVR
LWEEGEELELVEKSCTHSEKTNRTLSYRTGLKITSLTEVVCGLDLCN (SEQ ID NO: 2):
Linker between Domain 1 (D1) and Domain 2:
QGNSGRAVTYSRSRYLE (SEQ ID NO: 3)
uPAR Fragment-1:
LWEEGEELELVEK (SEQ ID NO: 4):
uPAR Domain 2 (D2):
CISCGSSDMSCERGRHQSLQCRSPEEQCLDVVTHWIQEGEEGRPKDDRHLRG
CGYLPGCPGSNGFHNNDTFHFLKCCNTTKCNE (SEQ ID NO: 5)
uPAR Fragment-2:
SPEEQCLDVVTHWIQEGEEGRPK (SEQ ID NO: 6)
uPAR Domain 3 (D3):
GPILELENLPQNGRQCYSCKGNSTHGCSSEETFLIDCRGPMNQCLVATGTHEP
KNQSYMVRGCATASMCQHAHLGDAFSMNHIDVSCCTKSGCNHPDLDVQYR (SEQ ID
NO: 7)
GPI Anchor:
SGAAPQPGPAHLSLTITLLMTARLWGGTLLWT (SEQ ID NO: 8)
Fragment 3/3:
GPILELENLPQNGR (SEQ ID NO: 9)
Fragment 4/4*:
RGPMNQCLVATGTHEPK (SEQ ID NO: 10)
Fragment 5:
SGCNHPDLDVQYR (SEQ ID NO: 11)

D2D3 protein with Linker: D2D3 protein lacks the D1 domain and the GPI anchor. See Barinka et al., “Structural Basis of Interaction between Urokinase-Type Plasminogen Activator and Its Receptor,” (2006) Mol Biol. 363 (2); pp. 482-495.

(SEQ ID NO: 12)
QGNSGRAVTYSRSRYLECISCGSSDMSCERGRHQSLQCRSPEEQCLDVV
THWIQEGEEGRPKDDRHLRGCGYLPGCPGSNGFHNNDTFHFLKCCNTTK
CNECNEGPILELENLPQNGRQCYSCKGNSTHGCSSEETFLIDCRGPMNQ
CLVATGTHEPKNQSYMVRGCATASMCQHAHLGDAFSMNHIDVSCCTKSG
CNHPDLDVQYR

The presence of D2D3 was examined by modifying immunoprecipitation (IP)-coupled to Western blot (WB) analysis originally developed for cancer patients. See Sidenius et al. 2000; Mustjoki et al., “Enhanced release of soluble urokinase receptor by endothelial cells in contact with peripheral blood cells,” (2000) FEBS Lett 486: pp. 237-242. As expected, high levels of suPAR were detected in DN patient sera. Importantly, proteins were identified that were similar in molecular weight to proteolytically generated human D2D3 (hD2D3) fragments in sera of a subset of DN patients (FIG. 1B). The inventors also detected hD2D3-like protein in the urine of DN patients (FIG. 1C), as seen previously in the urine of cancer patients. See Mustjoki et al., “Soluble urokinase receptor levels correlate with number of circulating tumor cells in acute myeloid leukemia and decrease rapidly during chemotherapy,” (2000) Cancer Res 60: pp. 7126-7132. However, hD2D3 was not detected in sera or urine from healthy individuals. See FIG. 5A: Sidenius et al. 2000; Mustjoki et al. 2000.

In order to demonstrate that the current IP-WB analysis was indeed detecting hD2D3 protein, mass spectrometry (MS) analysis on proteins immunoprecipitated from serum samples pooled from fourteen different DN patients were performed (FIG. 5B). MS identified a suPAR domain 2-specific peptide in the protein band corresponding to the D2D3 protein's molecular weight (FIG. 5B).

### Example 2

hD2D3 in Diabetic Neuropathy Patients With or Without Insulin Therapy

Diabetic neuropathy (DN) is a consequence of type-1 and type-2 diabetes. In addition, a growing number of adults are diagnosed with late-onset insulin-dependent diabetes, often referred to as Latent Autoimmune Diabetes in Adults (LADA). See Pozzilli et al., “Latent Autoimmune Diabetes in Adults: Current Status and New Horizons,” (2018) Endocrinol Metab (Seoul) 33: pp. 147-159. As type-1 diabetes and LADA in adults are highly heterogeneous and are often misdiagnosed as type-2 diabetes, the inventors focused on DN patients who were either receiving or not receiving insulin therapy and investigated whether hD2D3 was preferentially present in one of the two patient populations.

Two groups of patients were analyzed matched in age, sex, glycated hemoglobin A1c (HgbA1c) levels, estimated glomerular filtration rate (eGFR), and urine albumin to creatinine ratio (MALB/Crc) (FIG. 1D). Circulating hD2D3 was present in 55% of DN patients requiring insulin therapy in contrast to only 20% of DN patients not requiring insulin therapy (FIG. 1F). In a logistic regression model adjusting for HgbA1c and suPAR, the presence of hD2D3 was associated with a 4.28-fold (95% CI [1.54-11.88]) increase in the odds of the patient being on insulin. The presence of the hD2D3 protein in serum was a good discriminator between DN patients on insulin and those not on insulin (area under the curve (AUC)=0.674) (FIG. 1E).

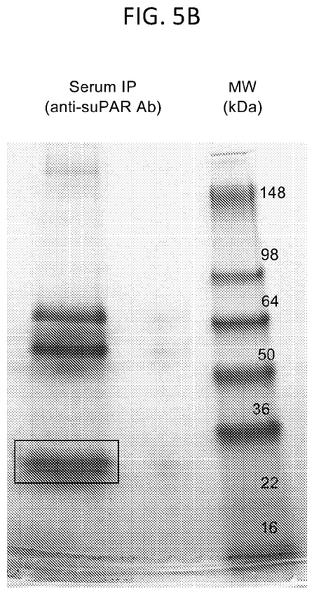

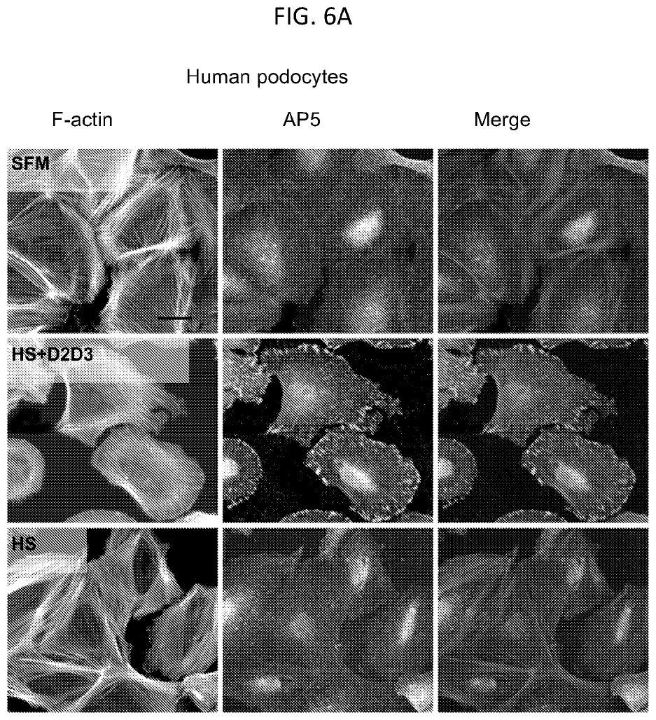

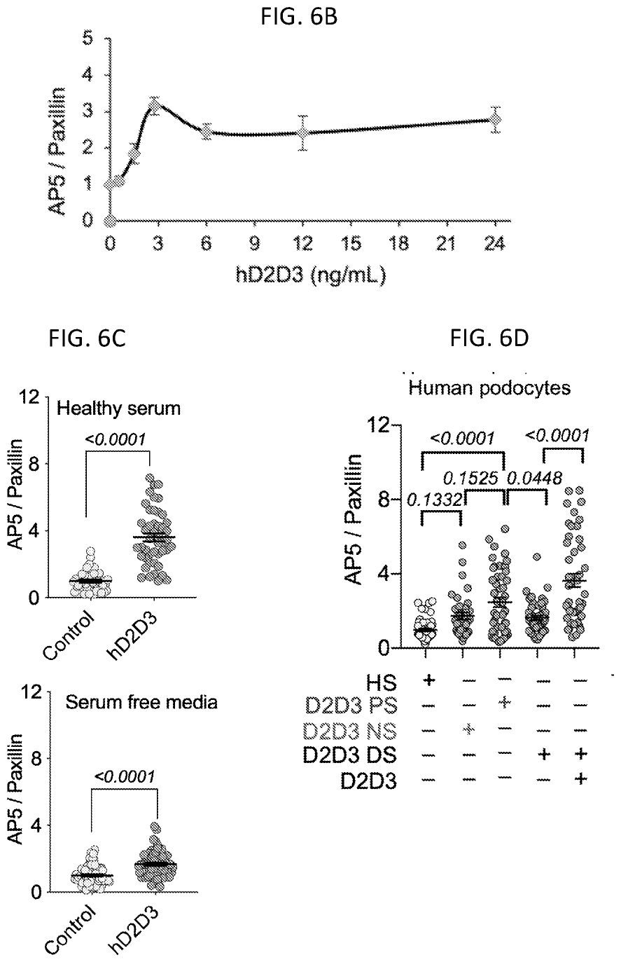

The inventors then elected to measure suPAR levels of the same patient population to investigate if the levels of D2D3 and full-length suPAR together can better distinguish between the insulin dependent and independent DN patients. They first expressed and purified recombinant hD2D3 (FIG. 6A, B,) and used it as a control to determine whether currently available suPAR ELISAs can detect hD2D3 protein.

FIG. 6A-D shows that recombinant hD2D3 protein activates β₃ integrin on human podocytes. (FIG. 6A) Representative images of human podocytes stained with phalloidin (F-actin) and AP5 (an antibody that recognizes active form of β₃ integrin). Notice that serum free media (SFM) and healthy serum (HS) do not activate β3 integrin, but that the addition of hD2D3 (2.5 ng/ml) to the HS resulted in AP5 signal. Scale bar, 5 μm. (FIG. 6B) Graph showing concentration-dependence of hD2D3's ability to activate β₃ integrin on human podocytes grown in the presence of healthy human serum. 20-101 cells were analyzed per treatment. Error bar=mean±SEM. Notice that at lower protein concentrations (1˜3 ng/ml) activation appears cooperative. (FIG. 6C) Scatter dot plots depicting ratio between activated β₃ integrin (AP5) and overall focal adhesions determined by the paxillin staining. Notice that addition of hD2D3 to SFM activated β3 integrin on human podocytes. This demonstrates that hD2D3 activates β₃ integrin on human podocytes even in the absence of any other serum proteins. For experiments with SFM, 62-79 cells were analyzed. For experiments with HS, 43-46 cells were analyzed. Data are shown as mean±SEM. An unpaired two-tailed/-test was performed to determine statistical significance (****P<0.0001). (FIG. 6D) Scatter dot plots showing levels of activated β₃ integrins on human podocytes (38-53 cells) treated with 10% healthy sera (HS), D2D3-positive sera (D2D3 PS), D2D3-negative sera (D2D3 NS), or D2D3 PS that were immunodepleted using an anti-uPAR antibody (R4) (D2D3 DS). Where indicated, 2.5 ng/ml hD2D3 was added. Data are presented relative to the control (HS or SFM). All results are presented as mean±SEM. Statistical significance was assessed using one-way ANOVA with Tukey's multiple comparisons.

Both commercially available ELISAs detected the recombinant hD2D3 protein in addition to full-length suPAR suggesting that these assays report, when present, a combined level of the fragment and the full-length protein in sera. Using the same logistic regression model, the inventors found that suPAR levels measured by ELISA together with the D2D3 protein identified using IP-WB had better discrimination ability than either one of them individually (FIG. 1E). Together. these data suggest that circulating hD2D3 could be a previously unrecognized molecular link between kidney disease and insulin-dependent diabetes.

### Example 3

Role of hD2D3 in Glucose-Stimulated Insulin Secretion by β Cells

It is well established that the loss of β-cell function and decreased glucose-stimulated insulin secretion (GSIS) underlie the pathogenesis of insulin-dependent diabetes. Thus, the inventors next examined whether the presence of hD2D3 in sera would inhibit the GSIS of human islets. To be able to perform multiple assays using an identical set of sera, sera was pooled from 8 DN patients on insulin therapy who contained hD2D3 protein (D2D3-positive sera) and 6 sera from DN patients who did not require insulin therapy (D2D3-negative sera). Compared to sera from healthy donors (HS), D2D3-positive sera impaired GSIS in human islets (FIG. 1G). This phenotype was reversed after hD2D3 and suPAR were immunodepleted using anti-uPAR antibody (D2D3 DS in the figure). Re-addition of recombinant hD2D3 but not suPAR resulted in impaired GSIS, convincingly demonstrating a direct role of hD2D3 in blocking insulin release by β-cells.

### Example 4

Role of hD2D3 in Podocyte Injury Following DN Glucose-Stimulated Insulin Secretion by β Cells

The inventors have previously shown that suPAR induces glomerular disease by activating αᵥβ₃ integrin on podocytes. See Hayek et al., “A tripartite complex of suPAR, APOL1 risk variants and αᵥβ₃ integrin on podocytes mediates chronic kidney disease.” (2017) Nat Med 23: pp. 945-953. Immunofluorescence (IF) microscopy was used to test whether the presence of hD2D3 would mediate the ability of sera to activate β₃ integrin on human podocytes. DN sera activated β₃ integrin regardless of the presence of hD2D3 when compared to the healthy sera (HS), possibly due to higher levels of suPAR (FIG. 1H). Immunodepleting suPAR and hD2D3 abolished the activating phenotype, which was re-established upon the addition of the hD2D3 protein (FIG. 1H). Similarly, the addition of hD2D3 converted non-activating HS into the activating serum in a concentration-dependent manner and was sufficient to induce β₃ integrin activation in serum-free media (SFM) (FIG. 6C, D). These observations implicate D2D3 in podocyte injury that underlies DN and suggest that D2D3 may directly injure two organ-specific cells, glomerular podocytes and pancreatic β-cells.

### Example 5

### Role of D2D3 Injury in a D2D3 Transgenic Mouse

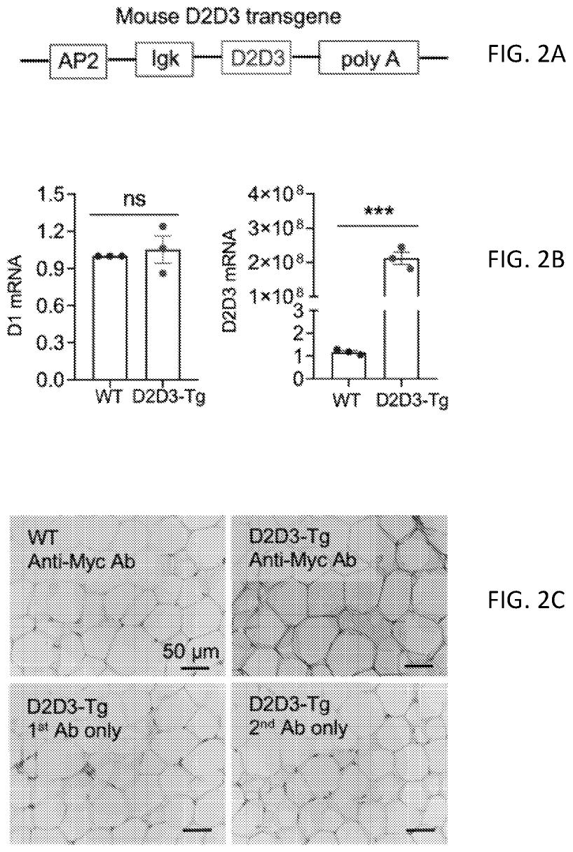

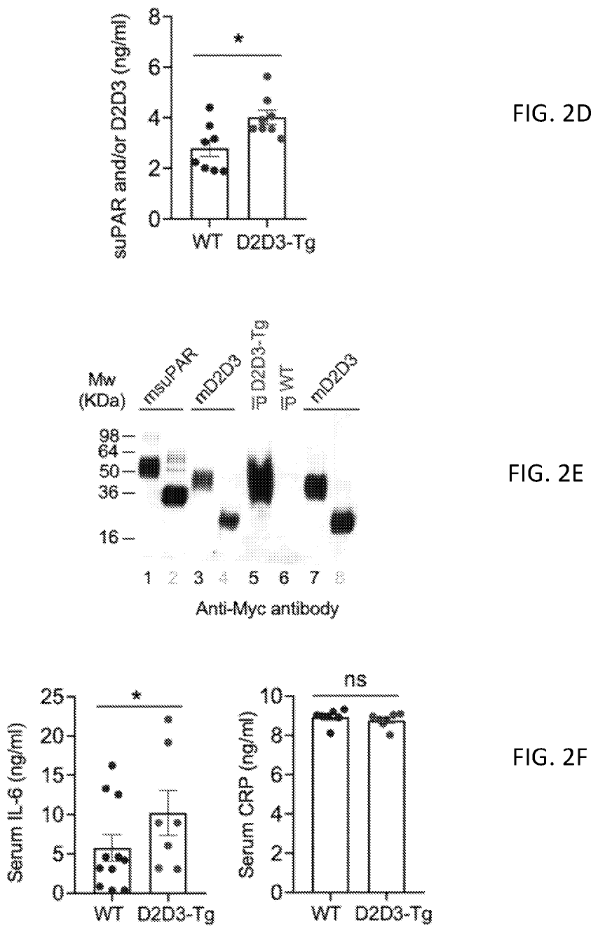

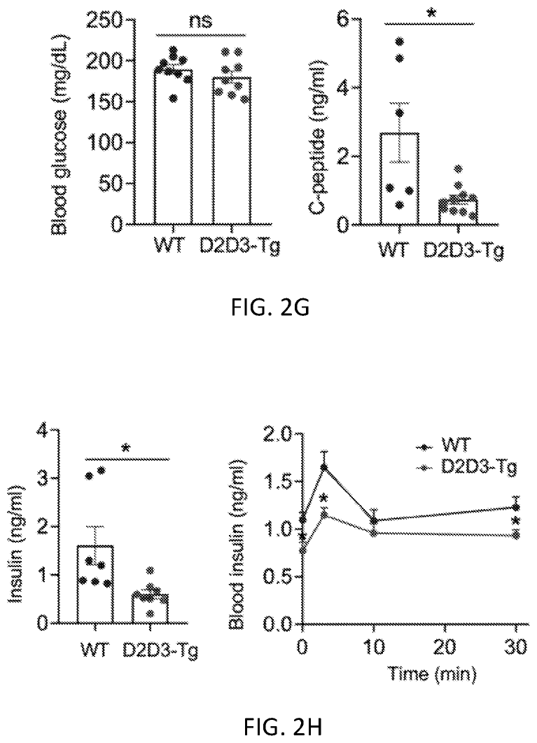

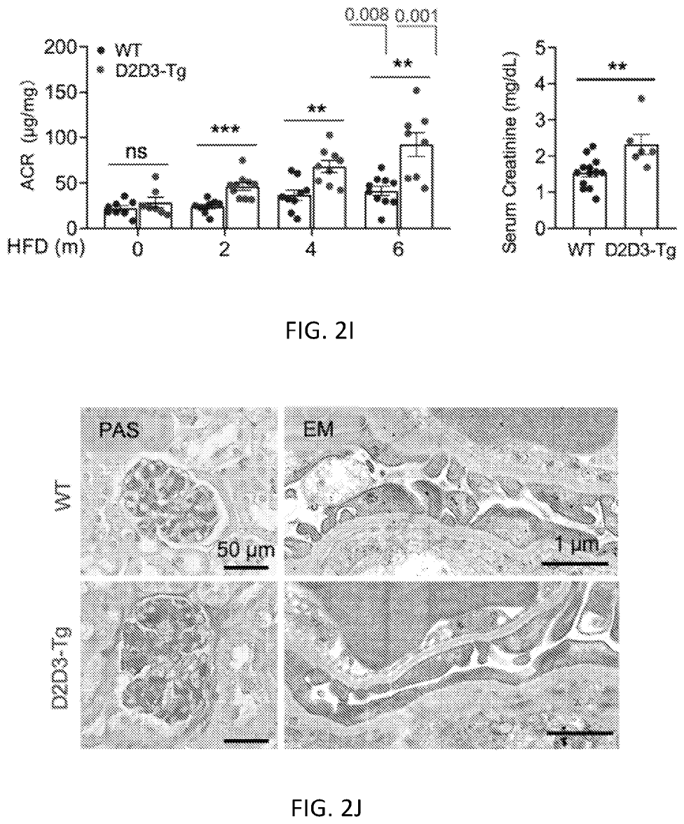

To test the hypothesis that D2D3 can injure both the glomerulus and the pancreas, the inventors generated a D2D3 transgenic mouse (D2D3-Tg) in which several studies were performed to validate their data. In the transgenic mouse model, the expression of Myc-tagged mouse D2D3 (mD2D3, FIG. 6B) was driven by the AP2 promoter (FIG. 2A).

FIG. 2A-J shows that D2D3-Tg mice present glomerular injury and insulin insufficiency. All animals were fed a high-fat diet (HFD) and measurements were performed on 6 months old animals unless stated differently. (FIG. 2A) Schematics of D2D3-Tg construction. (FIG. 2B) qPCR analysis of D2D3 and D1 in fat tissues normalized to Gapdh (n=3 biological replicates). (FIG. 2C) D2D3 detection in fat tissue of D2D3-Tg mice using anti-Myc antibody (D2D3 is Myc tagged). Controls: tissues stained with primary or secondary antibody only. (FIG. 2D) Mouse serum suPAR and D2D3 protein levels were determined using uPAR-specific ELISA (n=8). (FIG. 2E) Detection of Myc-tagged D2D3 in mouse sera. Sera were IPed with anti-uPAR antibody and detected by WB using anti-Myc antibody. Control: recombinant Myc-tagged mD2D3 (Lane 3, 4, 7, 8; +/−N-glycanase treated). (FIG. 2F) Serum IL-6 (WT. n=11: D2D3-Tg, n=7) and CPR (n=7, each group) levels. (FIG. 2G) Fasting blood glucose (n=9, each group), C-peptide (n=6 for WT; n=10 for D2D3-Tg), and insulin (n=7 for WT; n=8 for D2D3-Tg) levels. (FIG. 2H) In vivo GSIS. Overnight fasted animals were intraperitoneally injected with glucose (2 g/kg body weight) and their blood insulin levels were measured by ELISA (n=6-8 for each group). (FIG. 2I) Scatter dot plots presenting albumin creatinine ratio (ACR) or serum creatinine (n=7-10 for each group) levels. (FIG. 2J) PAS-stained kidney (left) and TEM of foot processes (right). (FIG. 2C, FIG. 2J). For all results, error bar, mean═SEM, (*P<0.05; **P<0.01; ***P<0.001. unpaired/-test). ns, not statistically significant.

FIG. 7A-C shows that ELISA detects mouse suPAR and mD2D3 protein. (FIG. 7A) Coomassie gel showing recombinant mouse suPAR and mD2D3 protein that were expressed and purified from HEK-293 cells. Notice that protein purification resulted in highly pure proteins. As both proteins are glycosylated. they travel as wide bands. (FIG. 7B) Table summarizing MWs of mouse proteins. MW of mD2D3 detected in the plasma of D2D3-Tg mice was similar to the MW of purified recombinant protein. (FIG. 7C) Mouse-specific ELISA (R&D) detected full length suPAR and mD2D3 equally well, but with low sensitivity BCA was used to determine protein concentrations used in ELISAs.

D2D3-Tg animals were viable, fertile, and born at a normal Mendelian ratio. RT-qPCR detected elevated mD2D3-specific mRNA levels in fat tissues with no alteration for the D1-specific mRNA levels (FIG. 2B). Immunohistochemistry using an anti-Myc antibody detected the presence of Myc-tagged mD2D3 in the fat tissue (FIG. 2C). The mouse suPAR-specific ELISA detected an approximately 2-fold increase in the suPAR levels in the sera of transgenic animals (FIG. 2D). To check if mouse suPAR-specific ELISA detected both mD2D3 and suPAR in sera, we expressed and purified recombinant mouse Myc-tagged suPAR and mD2D3 as controls (FIG. 7A-C). Indeed, both suPAR and mD2D3 were detected by ELISA, albeit with lower sensitivity (FIG. 7D). To identify and quantify the level of mD2D3 in circulation, IP-WB was performed and detected mD2D3 using an anti-Myc antibody. mD2D3 was detected at a concentration of 12±8 ng/ml only in the sera of D2D3-Tg, but not in control animals (FIG. 2E).

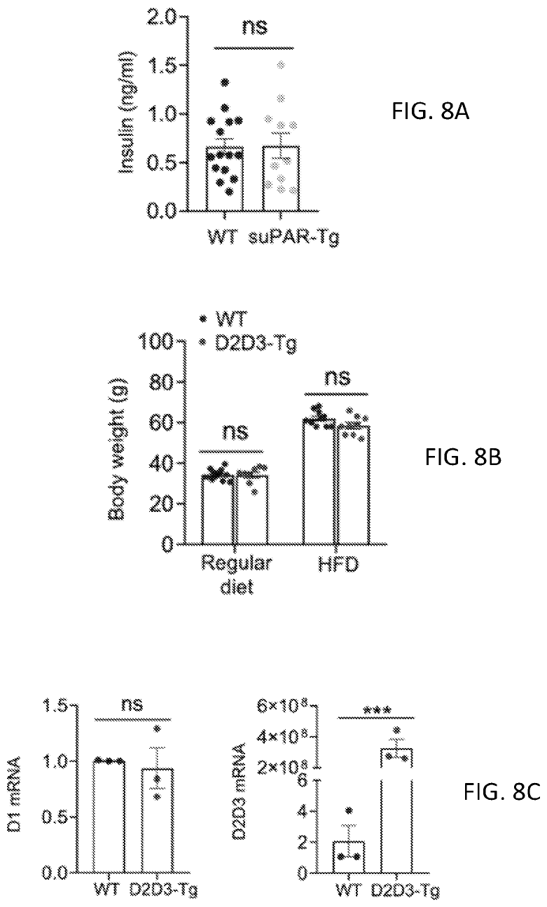

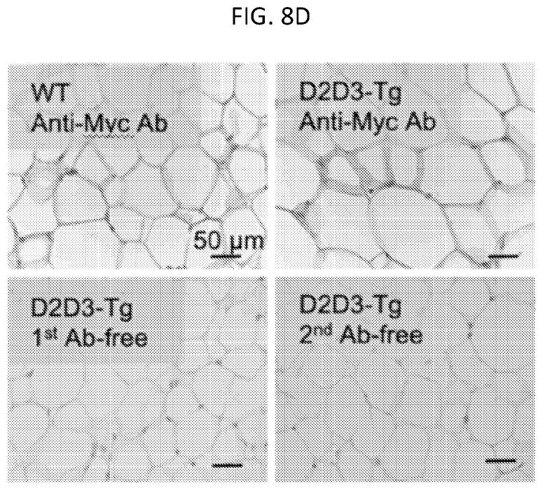

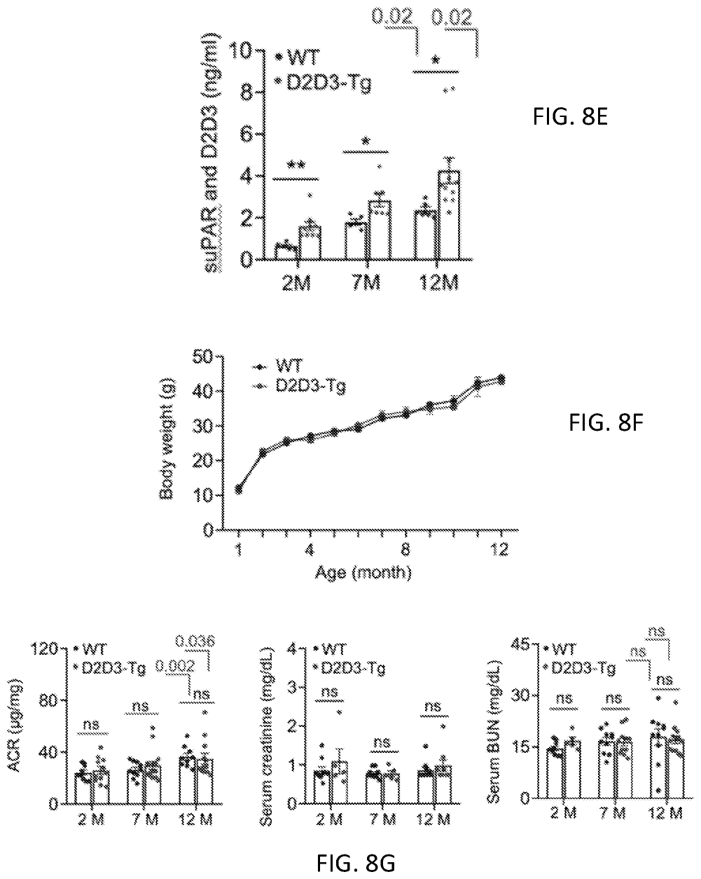

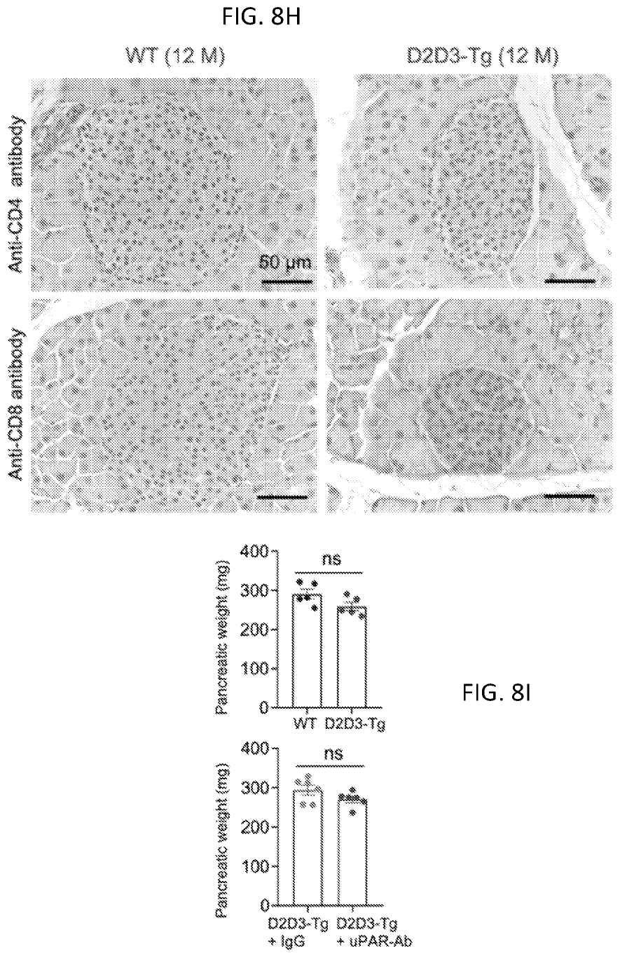

In line with the presence of hD2D3 protein in sera from DN patients, D2D3-Tg animals exhibited higher levels of IL-6 (FIG. 2F), a pro-inflammatory cytokine often increased in patients with diabetes. See Wegner et al., “Association between IL-6 concentration and diabetes-related variables in DM1 patients with and without microvascular complications,” (2013) Inflammation 36: pp. 723-728. Although levels of serum CRP or blood glucose were not significantly increased (FIG. 2F), D2D3-Tg animals exhibited lower levels of insulin and C-peptide suggesting impaired pancreatic b-cell function (FIG. 2G). As observed using D2D3-positive human sera and human islets. D2D3-Tg mice exhibited impaired GSIS (FIG. 2H). This phenotype was unique for mD2D3, as transgenic mice overexpressing full-length mouse suPAR (suPAR-Tg) (see Wei et al., “uPAR isoform 2 forms a dimer and induces severe kidney disease in mice,” (2019) J Clin Invest 129: 1946-1959) did not exhibit decreased levels of insulin (FIG. 8A). These data further confirm the role of the D2D3 protein in inhibiting b-cell function.

FIG. 8A-I shows that D2D3-Tg mice on a regular diet do not develop glomerular injury. (FIG. 8A) Scatter dot plot showing fasting blood insulin levels in WT and mice expressing mouse suPAR isoform 1 (suPAR-Tg) (n=15 for each group). (FIG. 8B) Scatter dot plots comparing body weights of animals kept on regular diet and on high fat diet (HFD). Notice that expression of mD2D3 had no effect on the body weight of the animals regardless of their diet. (FIG. 8C) qPCR analysis of D2D3 and DI in fat tissues. D2D3 and D1 mRNA levels were normalized to Gupdh mRNA level (n=3 biologic replicates). Data show that mRNA for D2D3 was detected in the fat tissue even when animals were fed regular diet. (FIG. 8D) mD2D3 protein is expressed in fat tissue of animals that were fed regular diet. As D2D3-Tg carries the c-Myc tag, immunohistochemistry was performed with a rabbit anti-Myc antibody. D2D3 was observed in adipocytes of D2D3-Tg mice, but not in control mice. Tissues stained with only primary or secondary antibody were used as negative controls. Scale bar, 50 μm. (FIG. 8E) Levels of suPAR and suPAR/D2D3 increased as animals aged. Scatter dot plots depicting protein levels determined using mouse-specific ELISA (n=5-11 for each group). (FIG. 8F) Body weight of WT and D2D3-Tg mice (n=5-8 for each group). (FIG. 8G) Kidney function was not affected in D2D3-Tg mice when they were fed regular diet. Kidney function was assessed by measuring ACR, serum creatinine and serum BUN levels (n=5-12 for each group). (FIG. 8H) Immunohistochemistry of islets using anti-CD4 and anti-CD8 antibodies in 12-months old animals. (FIG. 8I) Scatter dot plots comparing pancreatic weight between WT (n=5) and D2D3-Tg (n=5), as well as D2D3-Tg animals treated with IgG (n=6) or anti uPAR-Ab (n=6). All results were expressed as mean±SEM. Statistical analysis was determined using 2-tailed unpaired Student's t test. *P<0.05; **P<0.01; ***P<0.001. ns, not significant.

Since it was observed that hD2D3 dependent activation of αᵥβ₃ integrin on human podocytes, the inventors investigated the renal health of D2D3-Tg mice. Indeed, D2D3-Tg animals developed glomerular injury similar to a pattern seen in DN: progressive microalbuminuria accompanied by foot process (FP) effacement, elevated serum creatinine, glomerular hypertrophy, and mesangial expansion (FIG. 2I and J). Together with the data using human sera, these studies provide compelling evidence for the direct role of proteolytic D2D3 protein in inducing glomerular injury by activating avb3 on podocytes, and insulin insufficiency by impairing the function of β-cells.

### Example 6

### Role of D2D3 Physiology in a D2D3 Transgenic Mice Fed a Normal Diet

The phenotypic analysis in FIG. 2 was performed on 6 months-old mice fed with a high-fat diet (HFD) that enabled the expression of higher levels of mD2D3. As HFD can induce chronic low-grade systemic inflammation (see Laurentius et al., “High-fat diet-induced obesity causes an inflammatory microenvironment in the kidneys of aging Long-Evans rats.” (2019) J Inflamm (Lond) 16: p. 14). the inventors next examined the effects of mD2D3 on animal physiology when animals were fed a regular diet. As expected, there was a significant difference in the bodyweight of animals on different diets (FIG. 8B). Even on a regular diet, D2D3-Tg mice expressed mD2D3 protein (FIG. 8C, D), with suPAR/D2D3 levels increasing as animals aged and gained weight (FIG. 8E, F). Interestingly, D2D3-Tg mice on a regular diet did not develop significant kidney injury phenotypes (FIG. 8G) in contrast to the DN type of glomerular injury on HFD. This suggests that either kidney injury requires the presence of higher levels of mD2D3 protein in circulation, or that mD2D3 protein needs to synergize with HFD to induce kidney injury.

### Example 7

### Role of Diet on D2D3 Pancreatic Phenotypes

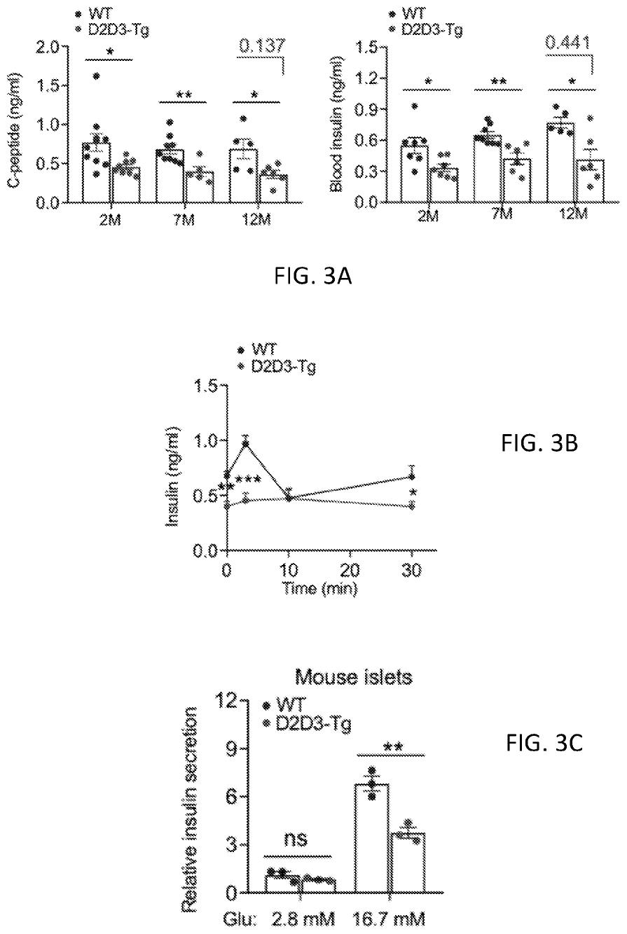

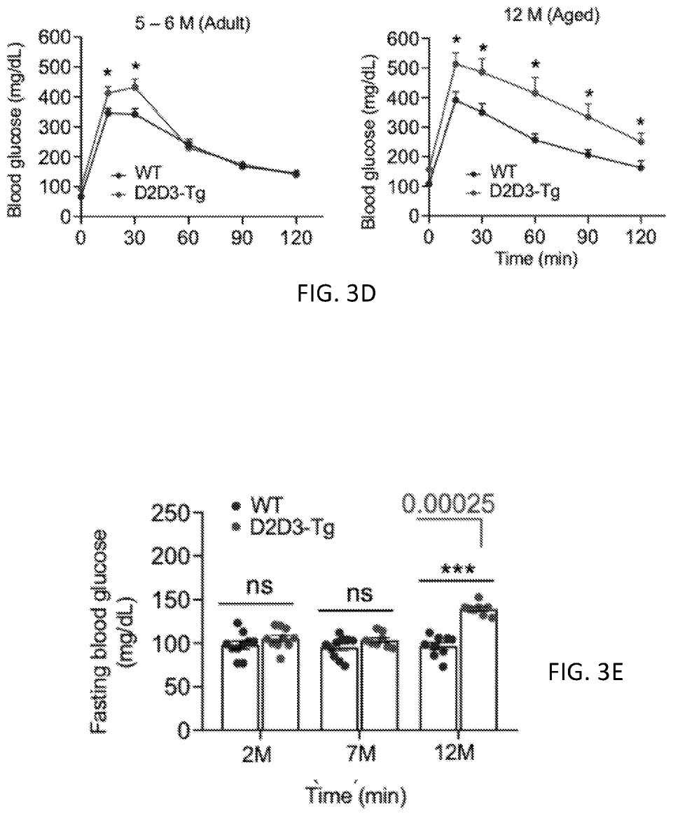

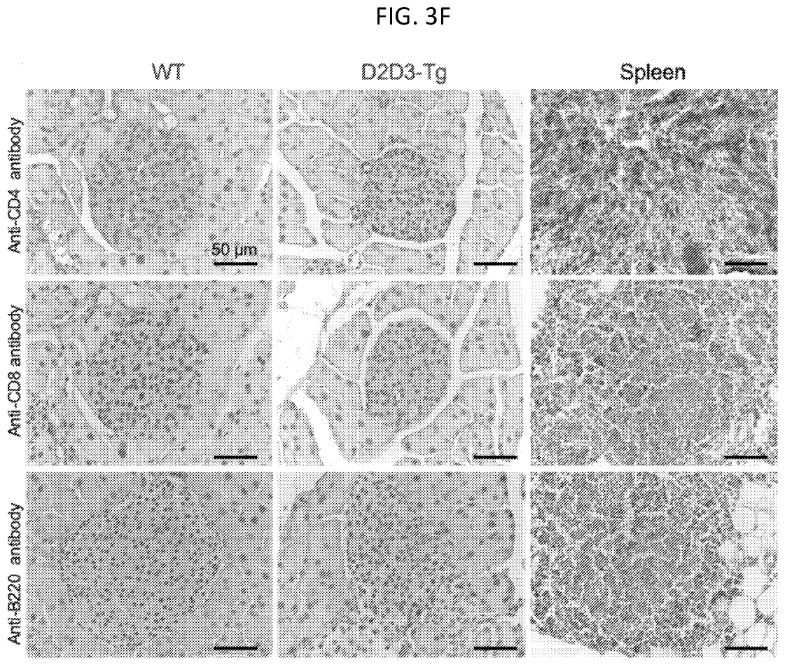

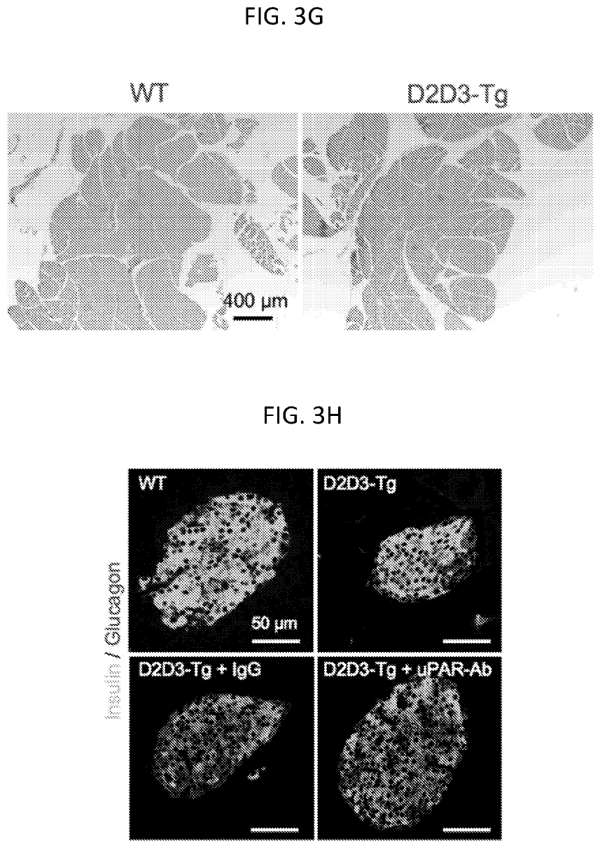

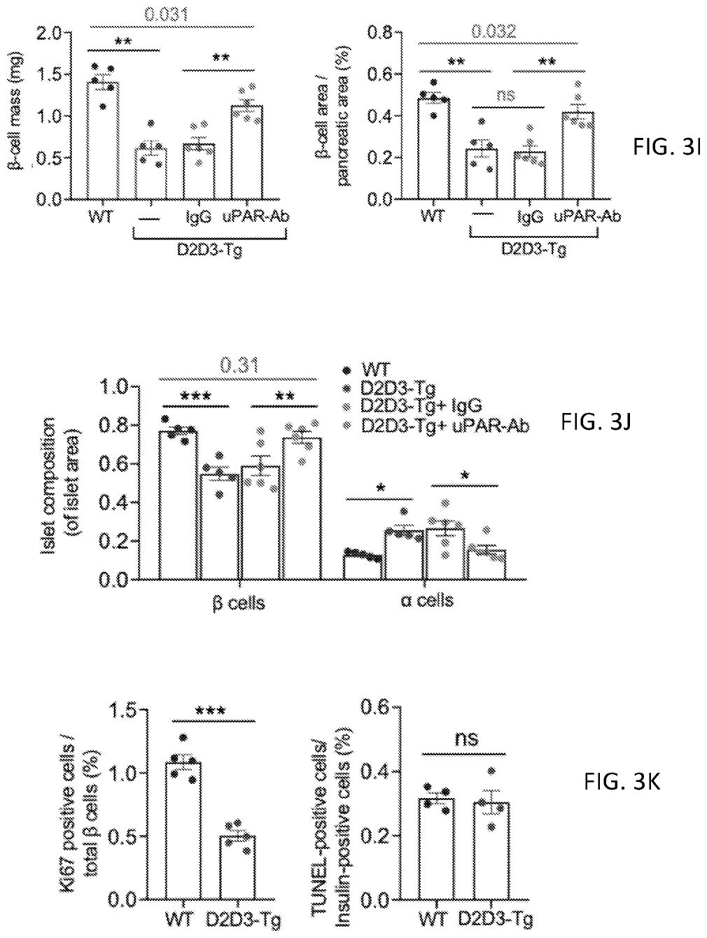

Although diet affected the D2D3-mediated kidney phenotype, it had no influence on the mD2D3-induced pancreatic phenotypes. D2D3-Tg mice fed with a regular diet exhibited decreased levels of C-peptide and blood insulin. as well as compromised in vivo GSIS even at an early age of 2 months (FIGS. 3, A and B).

FIG. 3A-N shows D2D3-Tg mice present insulin-dependent diabetes due to impaired pancreas function and β-cell mass. All animals were fed a regular diet. (FIG. 3A) Scatter dot plots representing fasting C-peptide and blood insulin levels (n=5-10 mice in each group) (FIG. 3B) In vivo GSIS. Overnight fasted animals were intraperitoneally injected with glucose (2 g/kg body weight) and their blood insulin levels were measured by ELISA (WT, n=5; D2D3-Tg, n=8). (FIG. 3C) In vitro GSIS. Islets from 2-month-old animals (n=3) were subjected to in vitro GSIS. (FIG. 3D) Glucose tolerance test (GTT). Adult (5-6 months) and aged (12 months) mice were fasted overnight before intraperitoneally injected with glucose (2 g/kg body weight). Blood glucose was measured at the indicated time points (n=5-6 animals per each condition). (FIG. 3E) Scatter dot plots depicting fasting blood glucose levels (n=8-10 mice per condition). (FIG. 3F) Representative immunohistochemistry of islets from 2-month-old mice stained with anti-CD4, anti-CD8, and anti-B220 antibodies. Spleen was used as a positive control for T and B cells staining (n=4 mice per each condition). (FIG. 3G) Representative immunohistochemistry of pancreas from 2-month-old mice stained with anti-insulin antibody (n=5 mice per each condition). (FIG. 3H) Representative immunohistochemistry of islets using anti-glucagon (α-cell) and anti-insulin (β-cell) antibodies (n=5 mice per each condition). (FIG. 3I) Scatter dot plots representing β-cell mass and β-cell area/pancreatic area ratio. Data were generated using images shown in (FIG. 3H). When indicated, D2D3-Tg mice were treated with anti-uPAR-Ab or IgG isotype control (IgG) for four weeks beginning at 2 months of age. Those data were collected at 3-month-old animals. (n=5 for WT and D2D3-Tg: n=6 for D2D3-Tg treated with either IgG or uPAR-Ab). Between 6-8 sections from each tissue were analyzed. (FIG. 3J) Scatter dot plots representing the composition of the islets in animals treated as described in (FIG. 3I). Islet composition was determined by counting the total number of β-cells (green) and α-cells (red) and expressing them as percentages of total cells counted within the single islet. (FIG. 3K) Scatter dot plots depicting levels of apoptosis determined by TUNEL staining, or cell proliferation determined by Ki67-positive staining of mice islets (n=5 mice for each condition). Each dot represents an average of 1000-1350 insulin-positive cells counted per animal. (FIG. 3L) In vivo GSIS using two months old D2D3-Tg treated with either IgG or anti-uPAR-Ab twice within one week. All animals were male (n=7-10 animals for each condition). (FIG. 3M, N) D2D3-Tg mice were treated with either anti-uPAR-Ab or IgG beginning at 2 months of age for four weeks. Experiments were performed at 3 months of age. All animals were male. Scatter dot plots representing blood insulin levels are shown in (FIG. 3M) (n=8 mice for each condition). The glucose tolerance test is shown in (FIG. 3N) (n=6 mice for each condition). For all results, error bar, mean±SEM. (*P<0.05: **P<0.01: ***P<0.001. unpaired t-test). ns, not statistically significant. In addition, when appropriate P numbers are shown in red or blue.

Islets isolated from the D2D3-Tg mice exhibited impaired ex-vivo GSIS (FIG. 3C), demonstrating that the observed phenotypes were inherent to the function of the pancreas. Concomitant with decreased insulin secretion, D2D3-Tg mice exhibited impaired glucose handling ability as evidenced glucose tolerance test (GTT) (FIG. 3D). Finally, the fasting blood glucose levels increased by 12 months of age (FIG. 3E). Together, these data show that D2D3-Tg mice develop insulin-dependent diabetes.

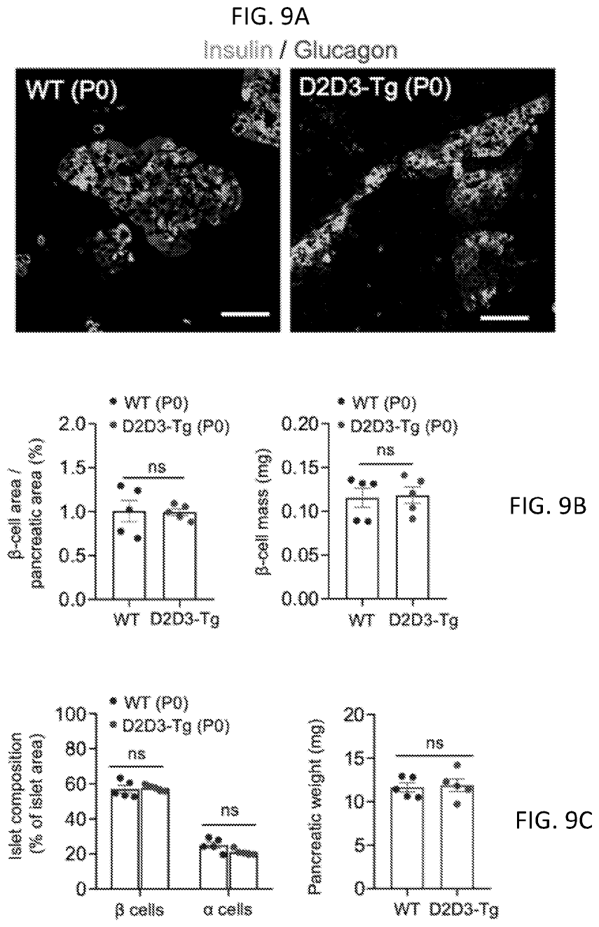

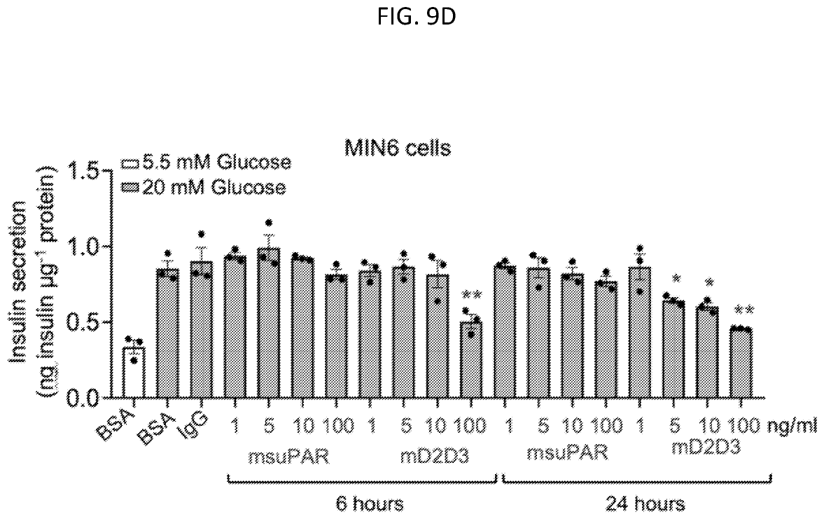

One of the characteristics of insulin-dependent diabetes is the reduction of pancreatic β-cells in part due to infiltration of autoreactive CD4 (+) and CD8 (+) T-cells. See Kelly et al., “Molecular aspects of type 1 diabetes,” (2003) Mol Pathol 56: pp. 1-10. Interestingly, D2D3-Tg animals did not exhibit major infiltration of CD4 (+), CD8 (+) T-cells, or B220 (+) B-cells either at 2-months of age (FIG. 3F) or at 12-months of age (FIG. 8H), indicating the involvement of a different mechanism that governs D2D3-mediated injury. Thus, the inventors further examined the islet architecture and cell distribution by staining the pancreas for insulin (β-cell marker) and glucagon (α-cell marker) (FIGS. 3G and H). Although, the D2D3-Tg animals did not exhibit decreased overall pancreatic weight (FIG. 8I), surprisingly, the inventors observed lower β-cell area and reduced β-cell mass (FIG. 3I). Additionally, a higher percentage of the α-cell population was observed, which altered the overall composition of the islets (FIG. 3J). Of note, these phenotypes were observed only in the post-natal animals, as neonatal wild type and D2D3-Tg mice exhibited similar distribution of pancreatic β-cells and α-cells (FIG. 9A-C).

FIG. 9A-D shows that neonatal D2D3-Tg mice have normal β-cell mass. (FIG. 9A) Immunohistochemistry of pancreas isolated from the neonatal mice (P0) stained with anti-insulin and anti-glucagon antibodies. (FIG. 9B) Staining shown in A was used to determine the β-cell area/pancreatic area ratio (n=5, for each group) as well as β-cell mass (n=5 for each group). (FIG. 9C) Scatter dot plots showing Islet composition (n=5, for each group) and pancreatic weight (n=5, for each group) in neonatal mice. (FIG. 9D) GSIS of MIN6 cells cultured in the presence or absence of BSA (100 ng/ml), IgG (100 ng/ml), mD2D3 protein or mouse suPAR (n=3). All results are expressed as mean±SEM. Statistical analysis was determined using 2-tailed unpaired Student's t test. *P<0.05: **P<0.01: ***P<0.001. ns, not significant.

Given that the reduced β-cell mass is only observed in post-neonatal D2D3-Tg animals, we next examined the proliferative capability of β-cells using Ki67 marker, as well as the level of apoptosis using TUNEL staining of the mouse islets. See Zhu et al., “Kindlin-2 modulates MafA and β-catenin expression to regulate β-cell function and mass in mice,” (2020) Nat Commun 11, 484. While no difference was found in the level of apoptotic cells in the islets of wild type and D2D3-Tg mice, β-cell proliferation was impaired in the D2D3-Tg mice (FIG. 3K). Together, these data demonstrate that the presence of D2D3 in circulation impairs β-cell proliferation, which leads to lower β-cell mass in postnatal D2D3-Tg mice in comparison to its wild-type counterpart.

### Example 8

### Role of Circulating D2D3 on Pancreatic β-Cells

To further affirm that the observed pancreatic injury phenotypes are a direct effect of D2D3, the inventors tested whether neutralizing the circulating D2D3 by anti-uPAR antibody (uPAR-Ab) can ameliorate the injury phenotypes. The inventors first treated 2 months old age-matched male D2D3-Tg mice twice within one week with either mouse anti-uPAR or IgG (control) antibody. Treatment with uPAR-Ab significantly restored the islet function in D2D3-Tg mice. demonstrated by improved in vivo GSIS (FIG. 3L). The inventors continued to treat mice twice a week for the following four weeks and observed that uPAR-Ab treated animals exhibited an increase in fasting blood insulin levels and improved glycemic excursion during glucose tolerance test in uPAR-Ab treated D2D3-Tg mice (FIG. 3M and N). The observed positive effect of uPAR-Ab on the function of the pancreas was due to improved β-cell area and mass as well as overall islet composition (FIG. 3I and J). Together, these data identify a novel mechanism that underlies insulin-dependent diabetes mellitus in which circulating D2D3 protein directly injures β-cells of the pancreas, and suggest that this pathogenic mechanism can be ameliorated by blocking D2D3 protein using specific anti-uPAR-Ab.

### Example 9

### Use of a Mouse Insulinoma Cell Line With Characteristics of Pancreatic β-Cells to Examine the Effects of D2D3

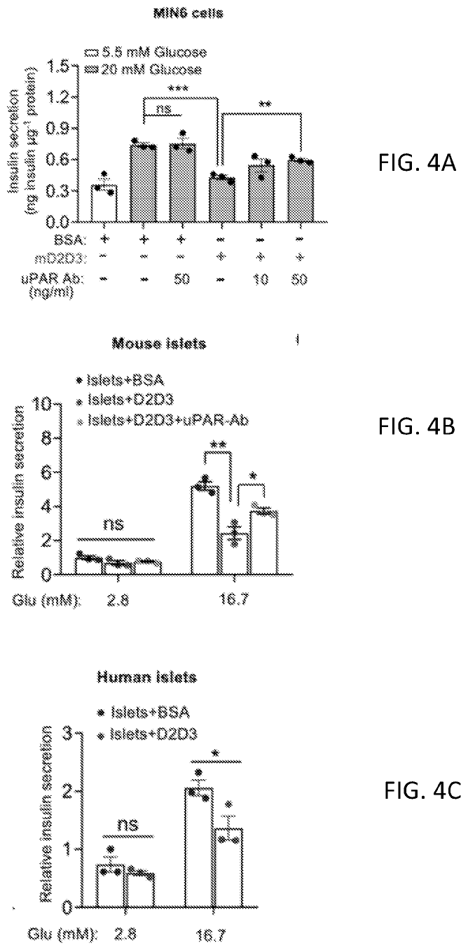

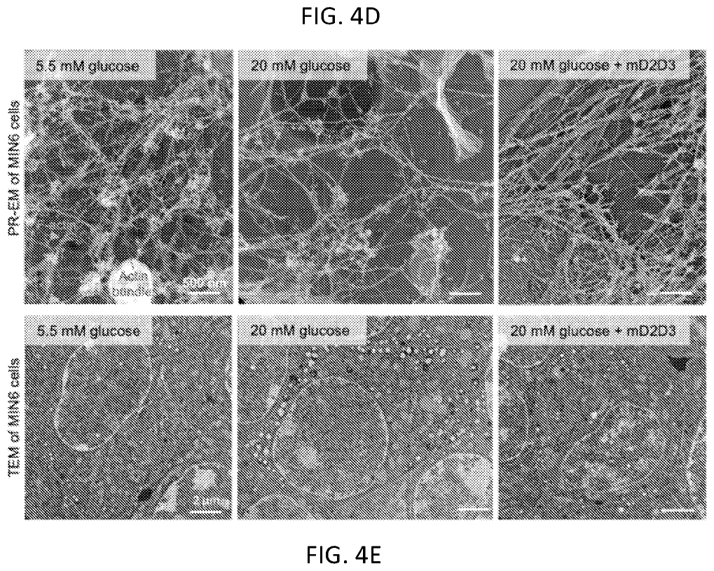

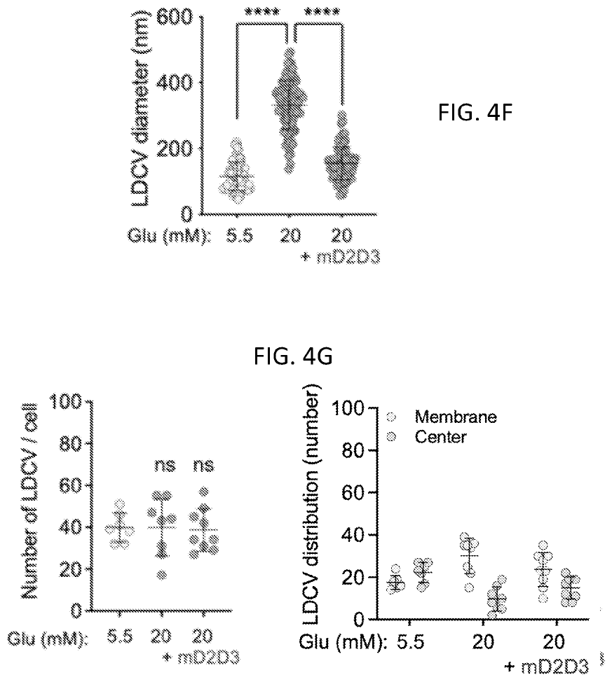

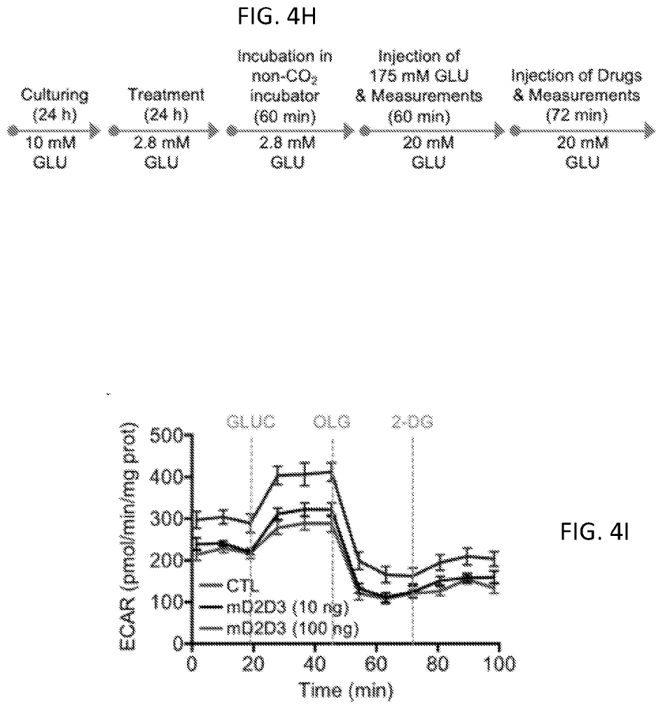

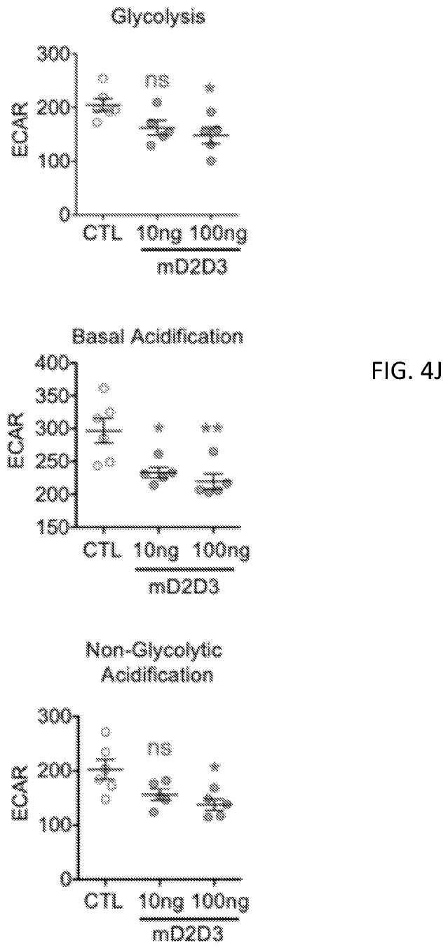

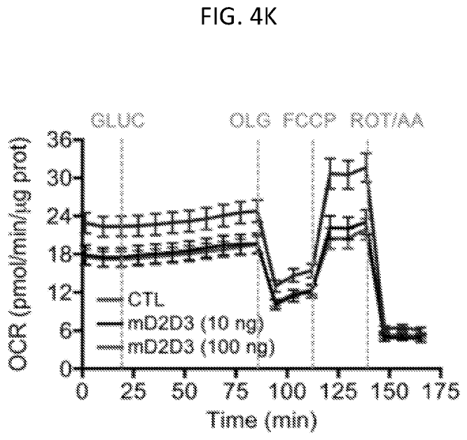

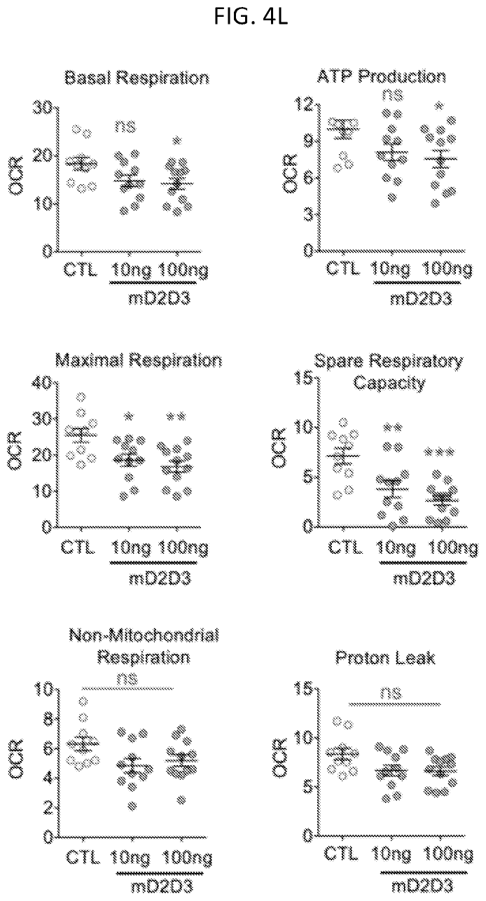

Next the inventors used MIN6 cells, a cell line derived from a mouse insulinoma with characteristics of pancreatic β-cells, to further elucidate the molecular mechanism by which D2D3 affects β-cell physiology. See Ishihara et al., “Pancreatic β-cell line MIN6 exhibits characteristics of glucose metabolism and glucose-stimulated insulin secretion similar to those of normal islets,” (1993) Diabetologia 36: pp. 1139-1145. The addition of recombinant mD2D3 impaired GSIS of MIN6 cells in a dose-and time-dependent manner (FIG. 9D), and this effect was inhibited by the addition of anti-uPAR-Ab (FIG. 4A). This effect was D2D3-specific since the addition of full-length suPAR did not inhibit GSIS (FIG. 9D). Identical trends were observed in isolated mouse islets, as well as human islets (FIG. 4B, C). Together, these data demonstrate that D2D3 directly impairs the ability of β-cells to release insulin upon glucose stimulation and establishes MIN6 cells as a relevant cell line for studying the effect of D2D3 on insulin secretion.

### Example 10

Determination of Which Steps in Insulin Release Were Inhibited by mD2D3

To determine which steps in insulin release were inhibited by mD2D3, the inventors next examined the effect of D2D3 had on the cytoskeleton using platinum replica electron microscopy (PR-EM) (FIG. 10A,B).

FIG. 10A,B mD2D3 show that fragment impairs glucose-stimulated reorganization of the actin cytoskeleton in MIN6 cells. (FIG. 10A) MIN6 cells were grown in low glucose (5 mM) before being stimulated by the addition of high glucose (20 mM) in the presence or absence of mD2D3 protein (100 ng/ml). The status of F-actin was examined by phalloidin staining. (FIG. 10B) Representative PR-EM micrographs of MIN6 cells focusing on the organization and the status of the actin cytoskeleton. Cells were grown as described in (FIG. 10A). Notice that mD2D3 inhibited high glucose-induced disassembly of actin filaments.

It is well documented that high glucose leads to microtubules (MT) polymerization, which aids the transport of secretory vesicles from the cell periphery to the plasma membrane, and simultaneous depolymerization of actin networks. See Wang et al., “Mechanisms of biphasic insulin-granule exocytosis-roles of the cytoskeleton. small GTPases and SNARE proteins.” (2009) J Cell Sci 122: pp. 893-903. Limited analysis has suggested that mD2D3 inhibited both processes: impaired MT polymerization and inhibited actin networks depolymerization (FIG. 4D).

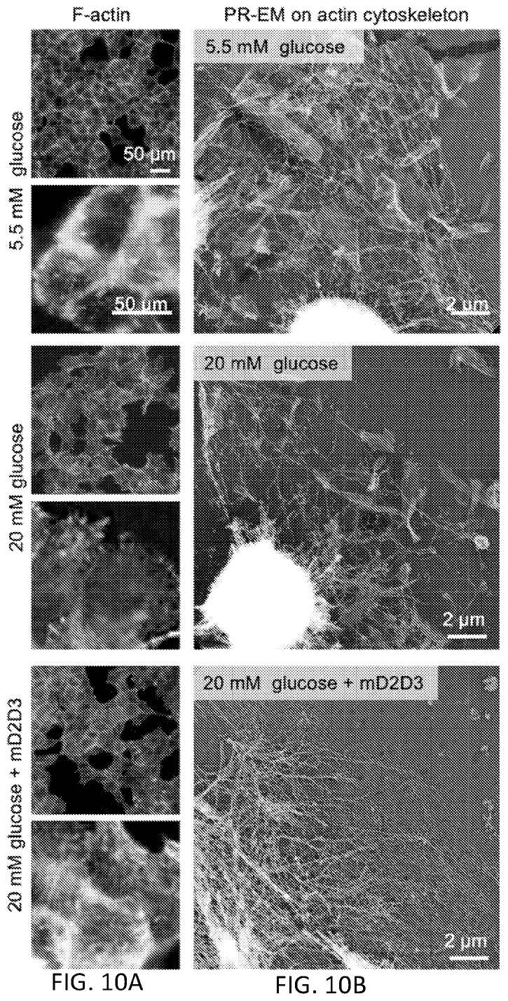

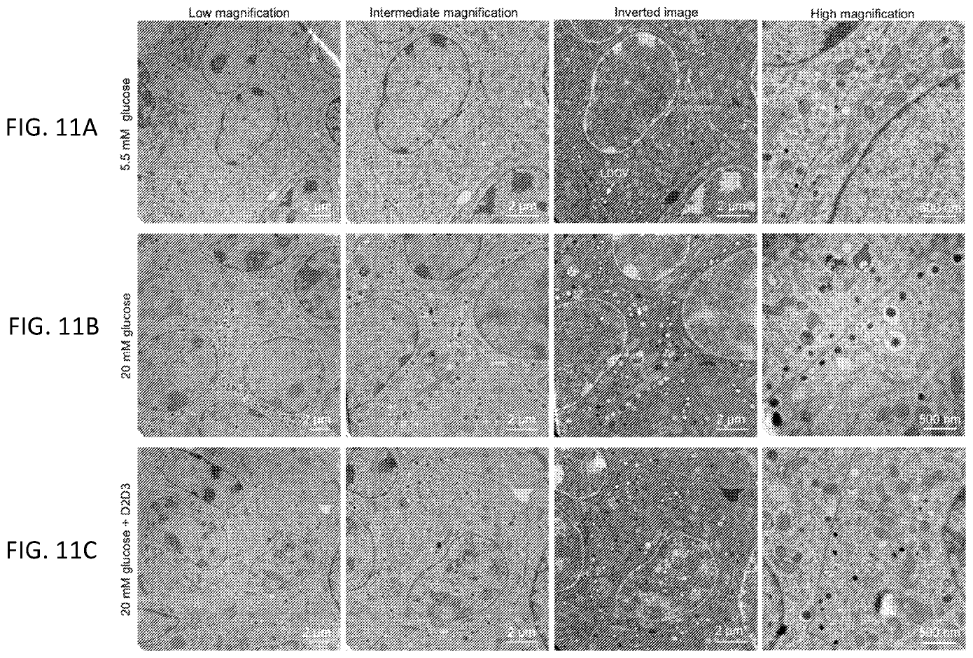

As dysregulated cytoskeletal dynamics is expected to have a profound effect on the trafficking and maturation of large dense-core vesicles (LDCVs) that transport insulin in β-cells, the inventors next visualized LDCVs using transmission electron microscopy (TEM) (FIG. 11A-C).

FIG. 11A-C shows that mD2D3 impairs insulin granule maturation and trafficking. Representative TEM micrographs of MIN6 cells. When indicated cells were grown in low glucose (5 mM) before stimulating by the addition of a high glucose (5 mM) in the presence or absence of mD2D3 protein (100 ng/ml) for 24 h. Images shown were captured at increasing magnifications. The plasma membrane is colored pink and large dense core vesicles (LDCVs) that transport insulin are colored yellow. Notice that LDCVs are smaller in size and less associated with the plasma membrane in MIN6 cells grown in the presence of the mD2D3 protein.

As seen in β-cells, glucose stimulation increased the diameter of LDCVs (vesicle maturation) as well as the number of membrane-associated LDCVs (vesicle trafficking) (FIG. 4E-G). See Ferri et al., “Insulin secretory granules labelled with phogrin-fluorescent proteins show alterations in size, mobility and responsiveness to glucose stimulation in living β-cells,” (2019) Sci Rep 9: p. 2890: Zhang et al., “Visualizing insulin vesicle neighborhoods in β-cells by cryo-electron tomography,” (2020) Sci Adv 6. Addition of mD2D3 impaired both of these processes demonstrating that the mD2D3 protein impairs multiple steps of insulin secretion by dysregulating glucose-induced cytoskeleton dynamics.

### Example 11

mD2D3 Effects on Glycolysis and Mitochondrial Respiration in MIN6 Cells Upon Glucose Stimulation

As insulin secretion is also directly coupled to glycolysis and oxidative metabolism (32, 33), we examined whether mD2D3 affected glycolysis and mitochondrial respiration in MIN6 cells upon glucose stimulation. See Rutter et al., “Pancreatic β-cell identity, glucose sensing and the control of insulin secretion,” (2015) Biochem J 466: pp. 203-218: Nicholls, “The Pancreatic β-Cell: A Bioenergetic Perspective,” (2016) Physiol Rev 96: pp. 1385-1447. To this end, extracellular acidification (ECAR) and oxygen consumption rates (OCR) were measured in real-time in MIN6 cells under basal conditions and in response to sequential injections of glycolysis or mitochondrial inhibitors using a Seahorse XFe24 extracellular flux analyzer. To mimic glucose stimulation, cells were grown at low glucose (2.8 mM) for 24 hours in the presence of increasing concentrations of D2D3 prior to switching to high glucose (20 mM) and initiation of the experiment (FIG. 4H). The presence of mD2D3 decreased glycolysis, basal acidification, and non-glycolytic acidification (FIG. 4I and J). In addition, mD2D3 decreased several bioenergetic parameters including basal and maximal respiration, spare respiratory capacity, and ATP production (FIG. 4K and L). Inability to increase OCR in response to glucose is expected to impair insulin granule maturation and trafficking as well as the proliferative capacity of β-cells. Therefore, these data identify a multi-consequential novel molecular mechanism by which a circulating pathogenic factor, D2D3, alters glucose-dependent β-cell physiology, including glycolysis, OCR, insulin granule maturation and trafficking, cytoskeletal dynamics, insulin secretion, and ultimately impairs β-cell proliferation in the post-natal period.

### Summary

Among the US population, 34.2 million people of all ages have diabetes. Although type-2 is the most prevalent form of diabetes, a growing number of adults are diagnosed with either type-1 diabetes or Latent Autoimmune Diabetes in Adults (LADA). Originally it was suggested that the main difference between type-1 diabetes and LADA is the age at which diseases present: type 1 diabetes commonly occurs in children, whereas LADA is diagnosed in adulthood. Recent data suggest that the main difference is not the age but the speed by which these diseases progress, thus implicating distinct molecular mechanisms for each disease. See Laugesen et al., “Danish Diabetes Academy, S. Workshop, Latent autoimmune diabetes of the adult: current knowledge and uncertainty,” (2015) Diabet Med 32: pp. 843-852: Jorns et al., “Pancreas Pathology of Latent Autoimmune Diabetes in Adults (LADA) in Patients and in a Rat Model Compared With Type 1Diabetes,” (2020) Diabetes 69: pp. 624-633. Despite these differences, a loss of pancreatic β-cells is a central feature of diabetes regardless of its etiology. Recently, signaling by the death receptor TMEM219 expressed on β-cells via its circulating ligand insulin-like growth factor binding protein 3 (IGFBP3) has been implicated in the loss of β-cells via induction of apoptosis. See D'Addio et al., “The IGFBP3/TMEM219 pathway regulates β-cell homeostasis,” (2022) Nat Commun 13: p. 684. It was suggested that blocking IGFBP3/TMEM219 signaling pathway might present a novel therapeutic option for both type-1 and type-2 diabetes. The current study identifies a novel molecular mechanism of diabetes in which a different circulating protein, the D2D3 protein of suPAR, causes injury to the β-cells by impairing insulin secretion and β-cell proliferation in the post-natal period via inhibition of glucose-dependent cell metabolism.

Furthermore, previous studies have pointed to β-cell-specific mechanisms of injury. See Id.; Levitsky et al., “Role of growth factors in control of pancreatic β-cell mass: focus on betatrophin,” (2014) Curr Opin Pediatr 26: pp. 475-479. In contrast, the current study shows that the D2D3 protein is capable of injuring two organs simultaneously. Treatment with uPAR-Ab attenuated D2D3-driven pancreatic injury in in vivo and ex vivo mouse models, and immunodepletion of hD2D3 from DN patient sera abated pathogenic phenotypes in human podocytes and islets. Thus, D2D3-specific antibodies offer a unique dual therapeutic option for chronic kidney disease and insulin-dependent diabetes.

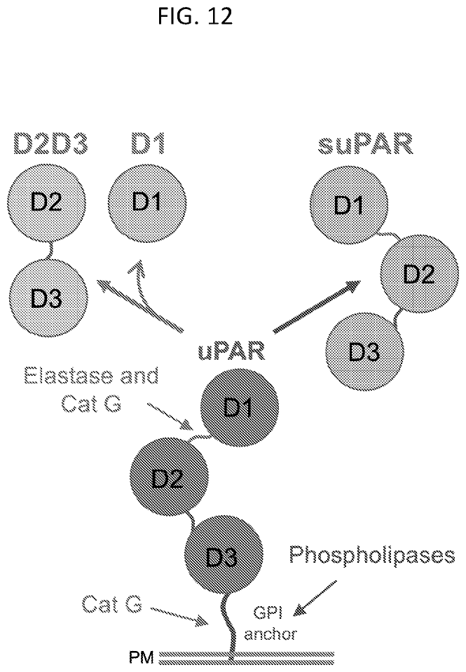

D2D3 protein is generated by the proteolysis of uPAR (see FIG. 12), which is an effector of the innate immune system as it is expressed by macrophages, neutrophils, and immature myeloid cells. See Hahm et al. 2017. Therefore, the study identifies a novel mechanism through which chronic inflammation leads to autoimmune diabetes mellitus. Given the central role of suPAR as a major risk factor in the COVID-19 associated kidney injury, as well as adverse outcomes in patients with diabetes mellitus, the current study warrants caution for the increased appearance of innate autoimmune diabetes in the post-COVID period globally. See Rovina et al., “Soluble urokinase plasminogen activator receptor (suPAR) as an early predictor of severe respiratory failure in patients with COVID-19 pneumonia,” (2020) Crit Care 24: p. 187; Azam et al., “Soluble Urokinase Receptor (SuPAR) in COVID-19-Related AKI,” J Am Soc Nephrol 31: pp. 2725-2735; Vasbinder et al., “Inflammation, Hyperglycemia, and Adverse Outcomes in Individuals with Diabetes Mellitus Hospitalized for COVID-19,” (2022) Diabetes Care 45: pp. 692-700.

As will be appreciated from the descriptions herein, a wide variety of aspects and embodiments are contemplated by the present disclosure, examples of which include, without limitation, the aspects and embodiments disclosed herein.

While embodiments of the present disclosure have been described herein, it is to be understood by those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of treating chronic kidney disease in a subject wherein the disease is characterized by the presence of detectable levels of the urokinase plasminogen activator receptor (uPAR) protein D2D3, the method comprising:
i) detecting the presence of D2D3 protein in a biological sample from the subject; and,
ii) administering a therapeutically effective amount of an agent that antagonizes and/or removes D2D3 from the circulation of the subject.

2. The method of claim 1, wherein the biological sample is selected from serum, plasma, saliva and urine.

3. The method of claim 1, wherein the agent comprises an anti-D2D3 antibody or antigen-binding fragment thereof that specifically binds to a D2D3 protein.

4. The method of claim 3, wherein the anti-D2D3 antibody or antibody fragment is humanized.

5. The method of claim 3, wherein the anti-D2D3 antibody or antibody fragment is a monoclonal antibody.

6. The method of claim 1, further comprising administration of an anti-soluble urokinase plasminogen activator receptor (suPAR) antibody or antibody fragment.

7. A method of treating insulin-dependent diabetes in a subject wherein the insulin-dependent diabetes is characterized by the presence of detectable levels of the urokinase plasminogen activator receptor (uPAR) protein D2D3, the method comprising:
i) detecting the presence of D2D3 protein in a biological sample from the subject; and,
ii) administering a therapeutically effective amount of an agent that antagonizes and/or removes D2D3 from the circulation of the subject.

8. The method of claim 7, wherein the biological sample is selected from serum, plasma, saliva and urine.

9. The method of claim 7, wherein the agent comprises an anti-D2D3 antibody or antigen-binding fragment thereof that specifically binds to a D2D3 protein.

10. The method of claim 9, wherein the anti-D2D3 antibody or antibody fragment is humanized.

11. The method of claim 9, wherein the anti-D2D3 antibody or antibody fragment is a monoclonal antibody.

12. The method of claim 7, further comprising administration of an anti-soluble urokinase plasminogen activator receptor (suPAR) antibody or antibody fragment.

13.-25 (canceled)

26. A composition comprising an agent that antagonizes the urokinase plasminogen activator receptor (uPAR) protein D2D3 in a therapeutically effective amount to treat a disease characterized by the presence of D2D3.

27. The composition of claim 26, wherein the anti-D2D3 antibody or antibody fragment D2D3 protein binds to a site on any one of SEQ ID NO: 1, SEQ ID NO: 3, SEQ ID NO: 5, SEQ ID NO: 6, SEQ ID NO: 7, SEQ ID NO: 8, SEQ ID NO: 10, SEQ ID NO: 11 and SEQ ID NO: 12.

28. (canceled)

29. (canceled)

30. (canceled)

31. (canceled)

32. (canceled)

33. (canceled)

34. (canceled)

35. (canceled)

36. (canceled)
